(19) United States
(12) Patent Application Publication
(10) Pub. No.: US 2026/0153798 A1
(43) Pub. Date: Jun. 4, 2026

(54) RESIST COMPOUND FOR PHOTOLITHOGRAPHY AND METHOD FOR MANUFACTURING SEMICONDUCTOR DEVICE USING THE SAME

(71) Applicant: Inha University Research and Business Foundation, Incheon (KR)

(72) Inventors: Jinkyun LEE, Incheon (KR); Gayoung KIM, Hwaseong-si (KR); Junsik KIM, Incheon (KR); Yejin KU, Incheon (KR); Sung Wook HWANG, Incheon (KR)

(73) Assignee: Inha University Research and Business Foundation, Incheon (KR)

(21) Appl. No.: 19/400,751

(22) Filed: Nov. 25, 2025

(30) Foreign Application Priority Data
Nov. 29, 2024 (KR) 10-2024-0174639
Nov. 18, 2025 (KR) 10-2025-0174301

(51) Int. Cl. G03F 7/004; C07C 43/215; C07C 43/23; C07F 7/22; G03F 7/00; G03F 7/09

(52) U.S. Cl. CPC G03F 7/0042; C07C 43/215; C07C 43/23; C07F 7/2208; G03F 7/094; C07C 2603/18; G03F 7/70033

(57) ABSTRACT

A resist compound for photolithography, includes an organic molecule, and the organic molecule includes a core structure containing at least six benzene rings and a functional group bonded to at least one of the benzene rings. The functional group is represented by Formula 1 or Formula 2.

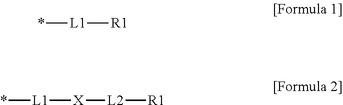

In Formulas 1 and 2 above, * is a portion bonded to a carbon of the at least one of the benzene rings, L1 and L2 are each independently an alkylene group having 1 to 10 carbon atoms, or oxygen, X is an alkylene group having 1 to 10 carbon atoms in which at least one hydrogen is substituted with a hydroxyl group (—OH), and R1 is an unsaturated hydrocarbon group having 2 to 10 carbon atoms with at least one C═C bond or at least one C≡C bond.

### CROSS-REFERENCE TO RELATED APPLICATIONS

This U.S. non-provisional patent application claims priority under 35 U.S.C. § 119 of Korean Patent Application Nos. 10-2024-0174639, filed on Nov. 29, 2024, and 10-2025-0174301, filed on Nov. 18, 2025, the entire contents of which are hereby incorporated by reference.

### BACKGROUND

The present disclosure herein relates to a resist compound for photolithography used for the manufacture of a semiconductor device, and a method for manufacturing a semiconductor device using the same.

This research was conducted with the support of Samsung Research Funding & Incubation Center for Future Technology (Project No. SRFC-TA1703-51, Research Project Title: Development of Resist and Application Process for Extreme UV Lithography (EUVL), Contribution Ratio: 8/10) and Dongjin Scholarship and Research Foundation (Research Project Title: Development of Tin-Containing Hybrid Resist for High NA Extreme UV Lithography; Contribution Ratio: 2/10).

Photolithography may include an exposure process and a development process. The exposure process may include irradiating a resist film with a specific wavelength of light to induce changes in chemical structure of the resist film. The development process may include selectively removing an exposed portion or an unexposed portion of the resist film by using a difference in solubility between the exposed portion and the unexposed portion.

Recently, as semiconductor devices are highly integrated and downsized, line widths of patterns in semiconductor devices become finer. To form fine patterns, a wide range of research is underway to enhance resolution and sensitivity of resist patterns formed by photolithography and to inhibit collapse of resist patterns. Furthermore, there is an increasing demand for resist patterns exhibiting superior etch resistance for an etching process.

### SUMMARY

The present disclosure provides a resist compound capable of improving the resolution and sensitivity of photoresist patterns, increasing the etch resistance of the photoresist patterns, and inhibiting the collapse of the photoresist patterns.

The present disclosure also provides a method for manufacturing a semiconductor device using the resist compound described above.

However, aspects of the present disclosure are not limited to the aforesaid, but other tasks not described herein will be clearly understood by those skilled in the art from descriptions below.

An embodiment of the inventive concept provides a resist compound for photolithography, including an organic molecule, wherein the organic molecule include a core structure containing at least six benzene rings and a functional group bonded to at least one of the benzene rings. The functional group is represented by Formula 1 or Formula 2.

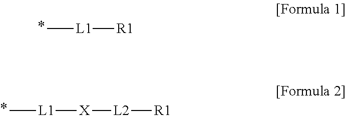

In Formulas 1 and 2 above, * is a portion bonded to a carbon of the at least one of the benzene rings, L1 and L2 are each independently an alkylene group having 1 to 10 carbon atoms, or oxygen, X is an alkylene group having 1 to 10 carbon atoms in which at least one hydrogen is substituted with a hydroxyl group (—OH), and R1 is an unsaturated hydrocarbon group having 2 to 10 carbon atoms with at least one C═C bond or at least one C≡C bond, or an unsaturated hydrocarbon group having 2 to 10 carbon atoms with at least one C═C bond and with at least one hydrogen substituted with a substituent represented by Formula 3,

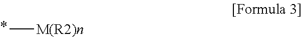

in Formula 3, * is a portion bonded to a carbon of R1, M is a metal, R2 is an aryl group having 6 to 30 carbon atoms, and n is an integer of 1 to 3.

In an embodiment of the inventive concept, a method for manufacturing a semiconductor device includes forming a photoresist film on a substrate. The photoresist film includes a resist compound containing an organic molecule. The organic molecule includes a core structure containing at least six benzene rings and a functional group bonded to at least one of the benzene rings. The functional group is represented by Formula 1 or Formula 2.

### BRIEF DESCRIPTION OF THE FIGURES

The accompanying drawings are included to provide a further understanding of the inventive concept, and are incorporated in and constitute a part of this specification. The drawings illustrate embodiments of the inventive concept and, together with the description, serve to explain principles of the inventive concept. In the drawings:

FIG. 1 is a view showing a ¹H NMR graph of HNF-propyne synthesized according to Example 1;

FIG. 2 is a view showing a mass spectrometry graph of HNF-propyne synthesized according to Example 1;

FIG. 3 is a scanning electron microscope image of a negative tone resist pattern formed by an e-beam lithography process according to Example 2;

FIG. 4 is a view showing a ¹H NMR graph of DMPF synthesized according to Example 3;

FIG. 5 is a view showing a ¹H NMR graph of DMPNF synthesized according to Example 3;

FIG. 6 is a view showing a mass spectrometry graph of DMPNF synthesized according to Example 3;

FIG. 7 is a view showing a 1H NMR graph of DHPNF synthesized according to Example 3;

FIG. 8 is a view showing a 1H NMR graph of Bis-FL synthesized according to Example 4;

FIG. 9 is a view showing a ¹H NMR graph of Bis-BPF synthesized according to Example 4;

FIG. 10 is a view showing a ¹H NMR graph of Bis-BNF synthesized according to Example 4;

FIG. 11 is a view showing a thermogravimetric analysis graph of HNF, DHPNF, and Bis-BNF according to Example 5;

FIG. 12 is a view showing a graph of differential scanning calorimetry analysis of HNF, DHPNF, and Bis-BNF according to Example 6;

FIG. 13 is a graph showing the results of measuring dry etch rates of HNF and DHPNF according to Example 7;

FIG. 14 is a view showing a ¹H NMR graph of DHPNF-propyne synthesized according to Example 8;

FIG. 15 is a scanning electron microscope image of a negative tone resist pattern formed by an e-beam lithography process according to Example 9;

FIG. 16 is a view showing a ¹H NMR graph of DHPNF-GPE synthesized according to Example 10;

FIG. 17 is a view showing a ¹H NMR graph of DHPNF-GAE synthesized according to Example 11;

FIG. 18 is a scanning electron microscope image of a negative tone resist pattern formed by an e-beam lithography process according to Example 12;

FIG. 19 is a graph showing the results of evaluating solubility of a resist thin film for a deep UV lithography process according to Example 13;

FIG. 20 is an optical microscope image of a negative tone resist pattern formed by a deep UV lithography process according to Example 13;

FIG. 21 is an optical microscope image of a negative tone resist pattern formed according to Example 15;

FIG. 22 is a scanning electron microscope image of a negative tone resist pattern formed by an e-beam lithography process according to Example 16;

FIG. 23 is a view showing a thermogravimetric analysis graph of HNF-propyne, DHPNF-propyne, DHPNF-GPE, and DHPNF-GAE according to Example 17;

FIG. 24 is a view showing a ¹H NMR graph of Bis-BPF-GPE synthesized according to Example 18;

FIG. 25 is a view showing a ¹H NMR graph of Bis-BPF-GAE synthesized according to Example 19;

FIG. 26 is a view showing a ¹H NMR graph of Bis-BNF-propyne synthesized according to Example 20;

FIG. 27 is an optical microscope image of a negative tone resist pattern formed according to Example 21;

FIG. 28 is a view showing a ¹H NMR graph of HSnPh₃ synthesized according to Example 22;

FIG. 29 is a view showing a ¹H NMR graph of HNF-propyne-HSnPh₃ synthesized according to Example 23;

FIG. 30 is a view showing a mass spectrometry graph of HNF-propyne-HSnPh₃ synthesized according to Example 23;

FIG. 31 is a view showing an elemental analysis graph of HNF-propyne-HSnPh₃ synthesized according to Example 23;

FIG. 32 is an optical microscope image of a negative tone resist pattern formed by a deep UV lithography process according to Example 24;

FIG. 33 is an optical microscope image of a negative tone resist pattern formed by a deep UV lithography process according to Example 25;

FIG. 34 is a scanning electron microscope image of a negative tone resist pattern formed by an e-beam lithography process according to Example 26;

FIG. 35 is a view showing a ¹H NMR graph of DMPNF-propyne synthesized according to Example 27;

FIG. 36 is a view showing a mass spectrometry graph of DMPNF-propyne synthesized according to Example 27;

FIG. 37 is a view showing a ¹H NMR graph of DMPNF-propyne-HSnPh₃ synthesized according to Example 28;

FIG. 38 is a view showing a mass spectrometry graph of DMPNF-propyne-HSnPh₃ synthesized according to Example 28;

FIG. 39 is a view showing an elemental analysis graph of DMPNF-propyne-HSnPh₃ synthesized according to Example 28;

FIG. 40 is an optical microscope image of a negative tone resist pattern formed by a deep UV lithography process according to Example 29;

FIG. 41 is a scanning electron microscope image of a negative tone resist pattern formed by an e-beam lithography process according to Example 30;

FIG. 42 is a graph showing the results of evaluating solubility of a resist thin film for an e-beam lithography process according to Example 31;

FIG. 43 is a graph showing the results of evaluating solubility of a resist thin film for an extreme UV lithography process according to Example 32; and

FIGS. 44 to 47 are cross-sectional views showing a method for manufacturing a semiconductor device using a resist composition according to embodiments of the inventive concept.

### DETAILED DESCRIPTION

In order to fully understand configuration and effects of the present disclosure, preferred embodiments of the present disclosure will be described with reference to the accompanying drawings. However, the present disclosure may be embodied in different forms and variously modified and changed, and should not be constructed as limited to embodiments set forth herein. Rather, these embodiments are provided so that the present disclosure will be thorough and complete, and will fully convey the scope of the present disclosure to those skilled in the art to which the invention pertains. Those skilled in the art will appreciate that the present disclosure may be carried out in a certain suitable environment.

Terms used herein are not for limiting the present disclosure but for describing embodiments. As used herein, singular terms are intended to include plural forms as well, unless the context clearly indicates otherwise. It will be further understood that the terms ‘comprises’ and/or ‘comprising’ as used herein specify the presence of stated elements, steps, operations, and/or components, but do not preclude the presence or addition of one or more other elements, steps, operations, and/or components.

As used herein, an alkyl group includes a monovalent saturated hydrocarbon group of a linear chain, branched chain, or cyclic chain, unless otherwise indicated.

As used herein, an alkylene group includes a divalent saturated hydrocarbon group of a linear chain, branched chain, or cyclic chain, unless otherwise indicated.

As used herein, an unsaturated hydrocarbon group includes a straight-chain or branched-chain hydrocarbon group containing one or more C═C bonds or one or more C≡C bonds, unless otherwise specified.

As used herein, the phrase “having a substituent” for a hydrocarbon group indicates that at least one of hydrogen atoms of the hydrocarbon group is substituted with a functional group or an atom, other than hydrogen atoms.

As used herein, a case of not drawing a chemical bond at a position where a chemical bond is required may indicate that a hydrogen atom is bonded at the position, unless otherwise defined.

Hereinafter, embodiments of the inventive concept are described in detail with reference to attached drawings. The same reference numerals are given for identical components in the drawings, and redundant descriptions thereof will be omitted.

A resist compound according to embodiments of the inventive concept will be described.

The resist compound according to embodiments of the inventive concept may be used for the manufacture of a semiconductor device and may be used in a photolithography process for the manufacture of a semiconductor device. The resist compound may be used, for example, in deep UV, extreme UV, or e-beam lithography processes. The deep UV may indicate ultraviolet having a wavelength of 160 nm to 280 nm. The extreme UV may indicate ultraviolet having a wavelength of 10 nm to 124 nm, particularly, a wavelength of 13.0 nm to 13.9 nm, and more particularly, a wavelength of 13.4 nm to 13.6 nm.

The resist compound may include an organic molecule. The organic molecule may include a core structure containing at least six benzene rings and a functional group bonded to at least one of the benzene rings. The functional group is represented by Formula 1 or Formula 2.

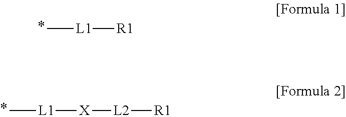

In Formulas 1 and 2,
* is a portion bonded to a carbon of the at least one of the benzene rings, L1 and L2 are each independently an alkylene group having 1 to 10 carbon atoms, or oxygen, X is an alkylene group having 1 to 10 carbon atoms in which at least one hydrogen is substituted with a hydroxyl group (—OH),
R1 is an unsaturated hydrocarbon group having 2 to 10 carbon atoms with at least one C═C bond or at least one C≡C bond, or an unsaturated hydrocarbon group having 2 to 10 carbon atoms with at least one C═C bond and with at least one hydrogen substituted with a substituent represented by Formula 3,

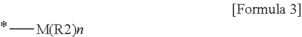

in Formula 3, * is a portion bonded to a carbon of R1, M is a metal, R2 is an aryl group having 6 to 30 carbon atoms, and n is an integer of 1 to 3.

In some embodiments, L1 and L2 may each independently be oxygen.

In some embodiments, R1 may be represented by Formula 4-1, Formula 4-2, or Formula 4-3.

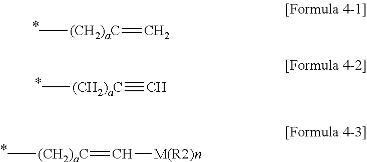

In Formulas 4-1 to 4-3, * is a portion bonded a carbon or an oxygen of, L1 of Formula 1 or L2 of Formula 2, and a is an integer of 1 to 8.

In Formula 4-3, M, R2, and n are as defined in Formula 3.

In Formulas 3 and 4-3, M may be one selected from the group consisting of tin (Sn), iodine (I), zinc (Zn), hafnium (Hf), zirconium (Zr), lithium (Li), sodium (Na), potassium (K), beryllium (Be), magnesium (Mg), calcium (Ca), barium (Ba), aluminum (Al), silicon (Si), cadmium (Cd), mercury (Hg), chromium (Cr), iron (Fe), cobalt (Co), nickel (Ni), copper (Cu), germanium (Ge), palladium (Pd), platinum (Pt), lead (Pb), strontium (Sr), and manganese (Mn).

According to some embodiments, the substituent represented by Formula 3 above may include a substituent represented by Formula 3-1.

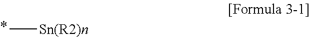

In Formula 3-1, * is a portion bonded to a carbon of R1, R2 is a phenyl group, and n is an integer of 1 to 3.

According to some embodiments, R1 may be a functional group represented by Formula 4-3-1.

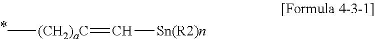

In Formula 4-3-1, * is a portion bonded to a carbon or an oxygen of, L1 of Formula 1 or L2 of Formula 2, a is an integer of 1 to 8, R2 is a phenyl group, and n is an integer of 1 to 3.

According to some embodiments, the resist compound may include an organic molecule represented by Formula 5.

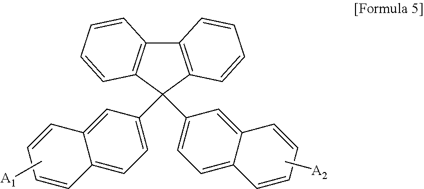

In Formula 5, A1 and A2 are each independently the functional group represented by Formula 1 or Formula 2.

According to some embodiments, A1 and A2 may each independently be a functional group represented by Formula 6-1, Formula 6-2, Formula 6-3, Formula 6-4, Formula 6-5, or Formula 6-6.

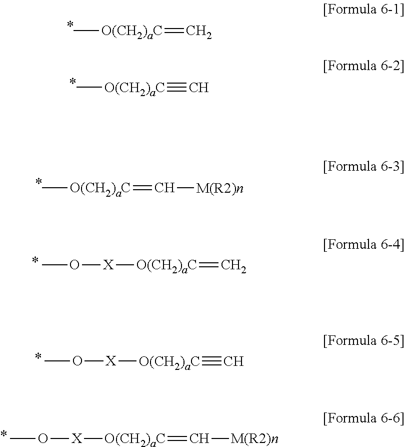

In Formulas 6-1 to 6-6, * is a portion bonded to a carbon of a benzene ring of Formula 5, and a is an integer of 1 to 8.

In Formula 6-3 and 6-6, M, as a metal, is at least one selected from the group consisting of tin (Sn), iodine (I), zinc (Zn), hafnium (Hf), zirconium (Zr), lithium (Li), sodium (Na), potassium (K), beryllium (Be), magnesium (Mg), calcium (Ca), barium (Ba), aluminum (Al), silicon (Si), cadmium (Cd), mercury (Hg), chromium (Cr), iron (Fe), cobalt (Co), nickel (Ni), copper (Cu), germanium (Ge), palladium (Pd), platinum (Pt), lead (Pb), strontium (Sr), and manganese (Mn), R2 is an aryl group having 6 to 30 carbon atoms, and n is an integer of 1 to 3.

In Formulas 6-4 to 6-6, X is an alkylene group having 1 to 10 carbon atoms, where at least one hydrogen is substituted with a hydroxyl group (—OH).

According to some embodiments, in Formulas 6-3 and 6-6, M may be tin (Sn), and R2 may be a phenyl group.

According to some embodiments, the resist compound may include an organic molecule represented by Formula 5-1 or Formula 5-2.

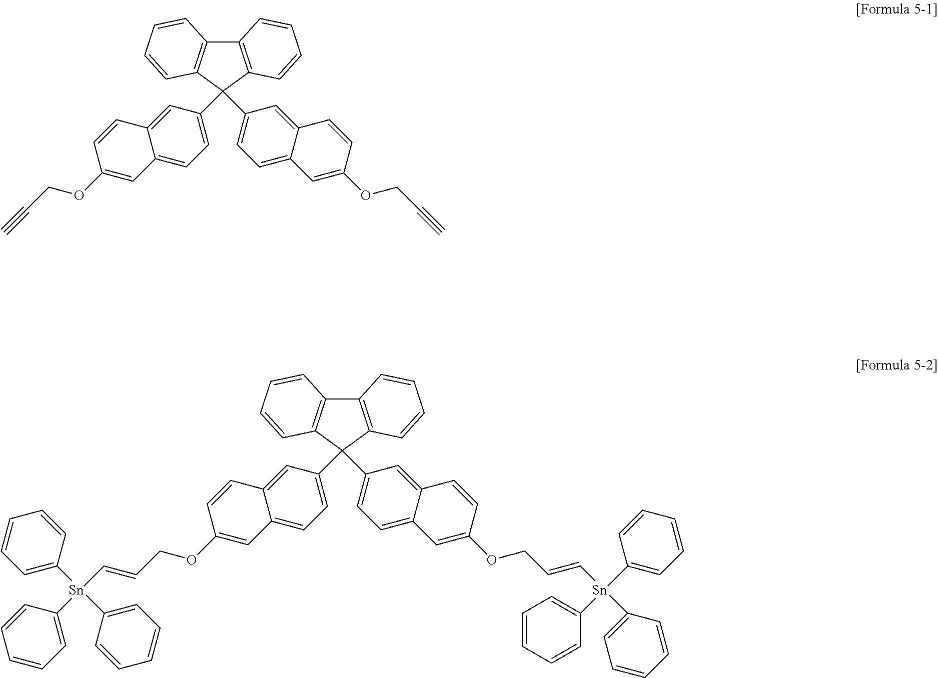 [SMILES: C#CCOc1ccc2cc(C3(c4ccc5cc(OCC#C)ccc5c4)c4ccccc4-c4ccccc43)ccc2c1 ; C(=[CH]\[Sn]([c]1ccccc1)([c]1ccccc1)[c]1ccccc1)/COc1ccc2cc(C3(c4ccc5cc(OC/C=[CH]/[Sn]([c]6ccccc6)([c]6ccccc6)[c]6ccccc6)ccc5c4)c4ccccc4-c4ccccc43)ccc2c1]

According to some embodiments, the resist compound may include an organic molecule represented by Formula 7.

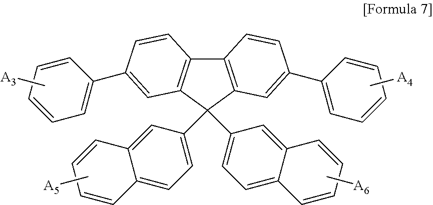

In Formula 7, A3 and A4 are each independently an alkoxy group having 1 to 10 carbon atoms or the functional group represented by Formula 1 or Formula 2, and A5 and A6 are each independently the functional group represented by Formula 1 or Formula 2.

According to some embodiments, A3 and A4 may each independently be an alkoxy group having 1 to 10 carbon atoms, or the functional group represented by Formula 6-1, Formula 6-2, Formula 6-3, Formula 6-4, Formula 6-5, or Formula 6-6, and A5 and A6 may each independently be the functional group represented by Formula 6-1, Formula 6-2, Formula 6-3, Formula 6-4, Formula 6-5, or Formula 6-6.

According to some embodiments, the resist compound may include an organic molecule represented by Formula 7-1, Formula 7-2, Formula 7-3, Formula 7-4, or Formula 7-5.

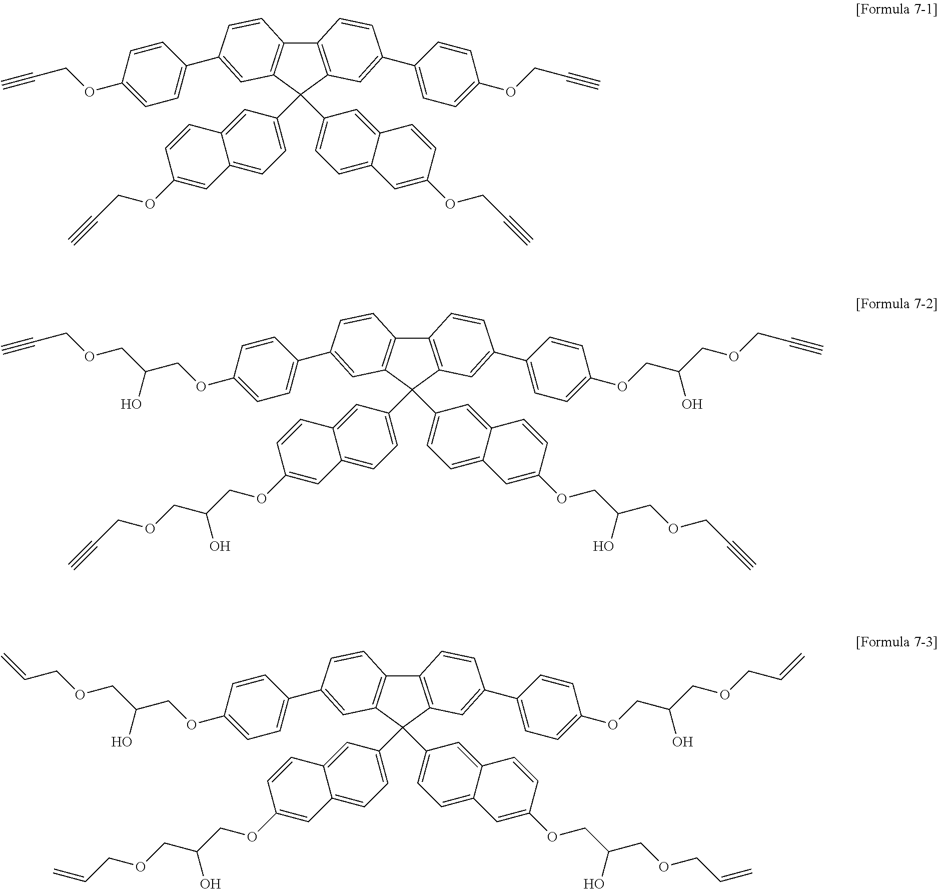

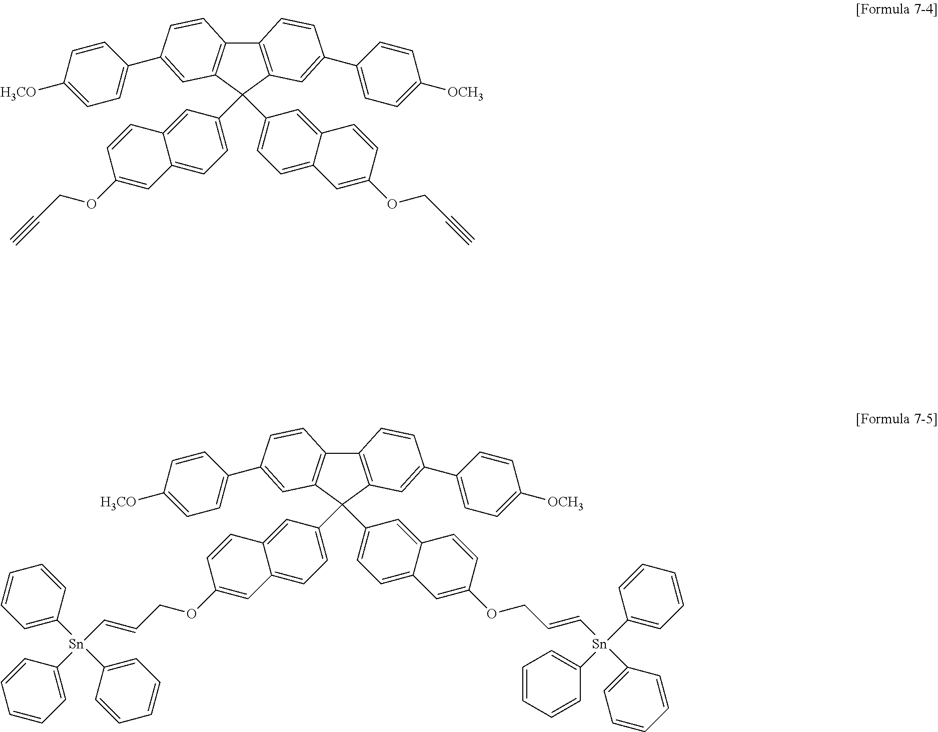 [SMILES: C#CCOc1ccc2cc(C3(c4ccc5cc(OCC#C)ccc5c4)c4cc(-c5ccc(OC)cc5)ccc4-c4ccc(-c5ccc(OC)cc5)cc43)ccc2c1 ; COc1ccc(-c2ccc3c(c2)C(c2ccc4cc(OC/C=[CH]/[Sn]([c]5ccccc5)([c]5ccccc5)[c]5ccccc5)ccc4c2)(c2ccc4cc(OC/C=[CH]/[Sn]([c]5ccccc5)([c]5ccccc5)[c]5ccccc5)ccc4c2)c2cc(-c4ccc(OC)cc4)ccc2-3)cc1]

According to some embodiments, the resist compound may include an organic molecule represented by Formula 8.

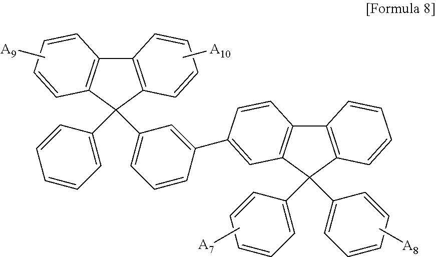

In Formula 8, A7, A8, A9, and A10 are each independently the functional group represented by Formula 1 or Formula 2.

In some embodiments, A7, A8, A9, and A10 may each independently be the functional group represented by Formula 6-1, Formula 6-2, Formula 6-3, Formula 6-4, Formula 6-5, or Formula 6-6.

In some embodiments, the resist compound may include an organic molecule represented by Formula 8-1, Formula 8-2, or Formula 8-3.

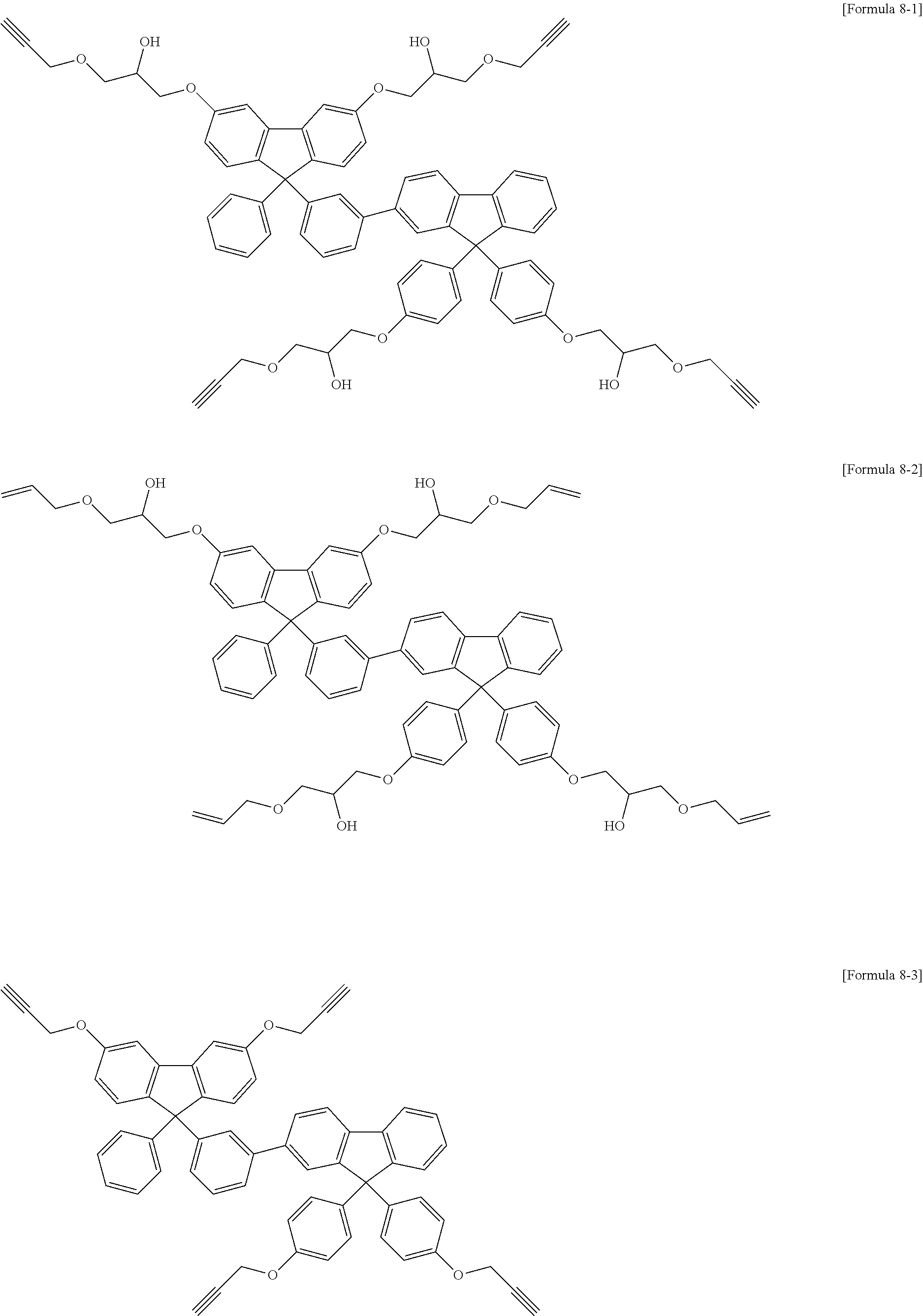

According to some embodiments, the resist compound may include an organic molecule represented by Formula 9.

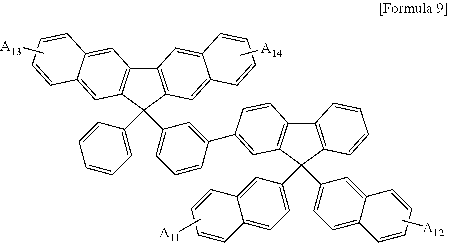

In Formula 9, A11, A12, A13, and A14 are each independently the functional group represented by Formula 1 or Formula 2.

In some embodiments, A11, A12, A13, and A14 may each independently be the functional group represented by Formula 6-1, Formula 6-2, Formula 6-3, Formula 6-4, Formula 6-5, or Formula 6-6.

According to some embodiments, the resist compound may include an organic molecule represented by Formula 9-1.

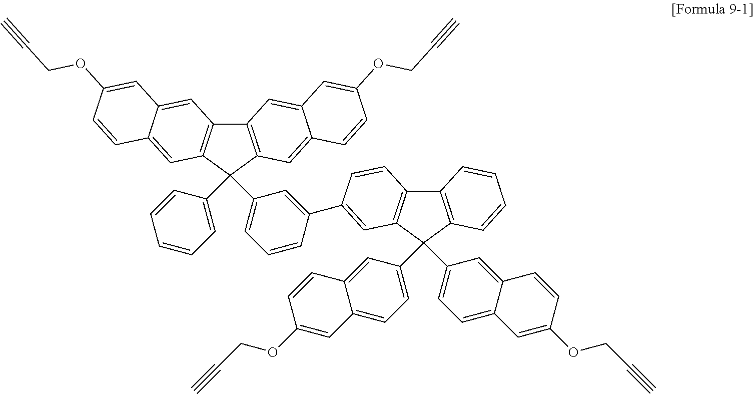 [SMILES: C#CCOc1ccc2cc(C3(c4ccc5cc(OCC#C)ccc5c4)c4ccccc4-c4ccc(-c5cccc(C6(c7ccccc7)c7cc8ccc(OCC#C)cc8cc7-c7cc8cc(OCC#C)ccc8cc76)c5)cc43)ccc2c1]

According to embodiments of the inventive concept, the resist compound may be a non-chemically amplified resist compound containing an organic molecule. The organic molecule may include a core structure containing at least six benzene rings and a functional group bonded to at least one of the benzene rings. The functional group may be represented by Formula 1 or Formula 2. When deep UV, e-beam, or extreme UV is applied to the resist compound, a C═C bond or a C≡C bond of an R1 group of the functional group represented by Formula 1 or Formula 2 may readily accept secondary electrons generated by photons, and accordingly, radicals (e.g., carbon radicals) may be generated. The organic molecules adjacent to each other may be cross-linked through a bond between the radicals, or a bond between the C═C bond or C≡C bond of the R1 group of the functional group and the radicals. Consequently, the resist compound may exhibit reduced solubility by irradiation with the light. Accordingly, when an exposure process is performed on a photoresist film formed using the resist compound, a difference in solubility between an exposed region and an unexposed region of the photoresist film may be induced.

According to an aspect of the inventive concept, the resist compound may include the organic molecule having a smaller molecular weight than that of a polymer, and thus photoresist patterns formed using the resist compound may exhibit reduced line width roughness and increased resolution. In addition, the organic molecule may include the core structure containing at least six benzene rings, and thus the photoresist film formed using the resist compound may exhibit increased etch resistance. Furthermore, the organic molecule may include the functional group represented by Formula 1 or Formula 2, and thus a C═C bond or a C≡C bond of an R1 group of the functional group may readily accept secondary electrons generated by photons, and accordingly, the photoresist film may exhibit increased sensitivity.

According to some embodiments, the R1 group of the functional group represented by Formula 1 or Formula 2 may have a substituent represented by Formula 3. In this case, the etch resistance of the photoresist film may be further increased by a metal element of the substituent. Moreover, the metal element (e.g., tin) may have high absorption characteristics for extreme UV, which may consequently lead to a further increase in the sensitivity of the photoresist film.

According to some embodiments, when the etch resistance of the photoresist film is increased, the photoresist film may be formed to be relatively thin, which consequently allows the photoresist patterns to be formed with a relatively small aspect ratio. Accordingly, pattern collapse of the photoresist patterns may be minimized during a development process.

### [Example 1] Synthesis of HNF-Propyne (Formula 5-1) Introduced with a Carbon-Carbon Triple Bond

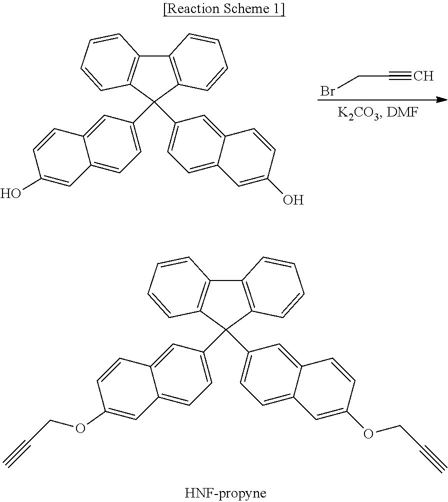

6,6′-(9H-fluorene-9,9-diyl)bis(naphthalen-2-ol) (0.50 g, 1.11 mmol), propargyl bromide (0.30 g, 2.55 mmol), and K₂CO₃ (0.46 g, 3.33 mmol) were added to a 100 cm³ round-bottom flask, and dimethylformamide (DMF, 5 cm³) was added as a solvent to form a reaction solution. The reaction solution was stirred at room temperature for 12 hours to form a reactant. The reactant was diluted using ethyl acetate (150 cm³). An organic solvent layer of the reactant was washed twice with water, and then washed once more using a saturated aqueous sodium chloride solution (150 cm³). Moisture contained in the organic solvent layer was then removed using anhydrous MgSO₄, and the organic solvent layer, after moisture removal, was concentrated under reduced pressure to form a product. The product was purified through column chromatography using chloroform as a mobile phase and then concentrated under reduced pressure to obtain HNF-propyne (0.66 g, 88%) as a white solid.

¹H NMR (400 MHz, CDCl₃): δ=7.84 (d, J=7.6 Hz, 2H, Ar—H), 7.68 (d, J=9.0 Hz, 2H, Ar—H), 7.58 (d, J=10.0 Hz, 4H, Ar—H), 7.52 (d, J=7.8 Hz, 2H, Ar—H), 7.44-7.39 (m, 4H, Ar—H), 7.32 (d, J=7.5 Hz, 2H, Ar—H), 7.22 (s, 2H, Ar—H), 7.14 (d, J=9.9 Hz, 2H, Ar—H), 4.80 (s, 4H, —OCH₂—), 2.54 (s, 2H, —C≡CH)/m/z (ESI-TOF-MS) 526.1927 ([M+H]⁺. C₃₉H₂₆O₂H requires M, 527.63).

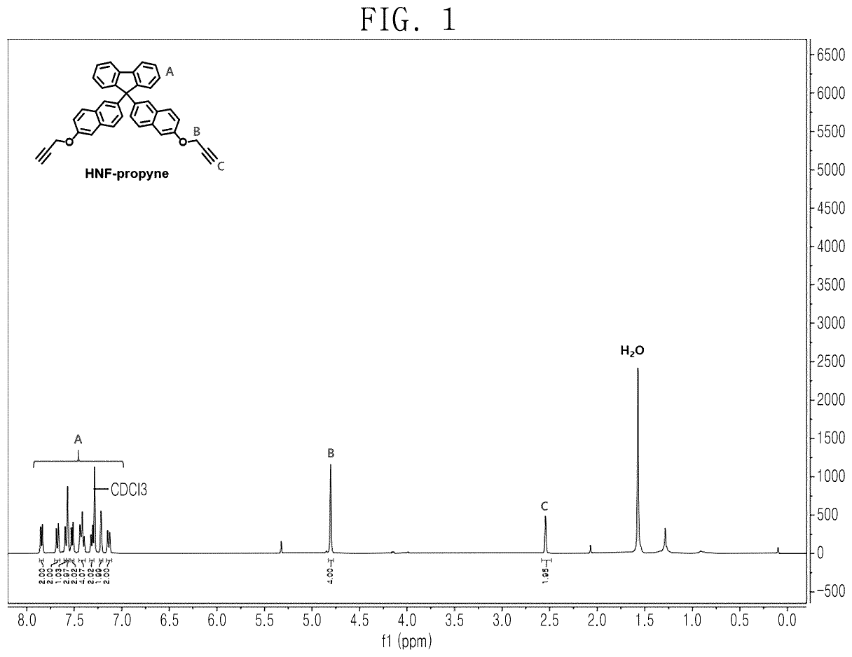

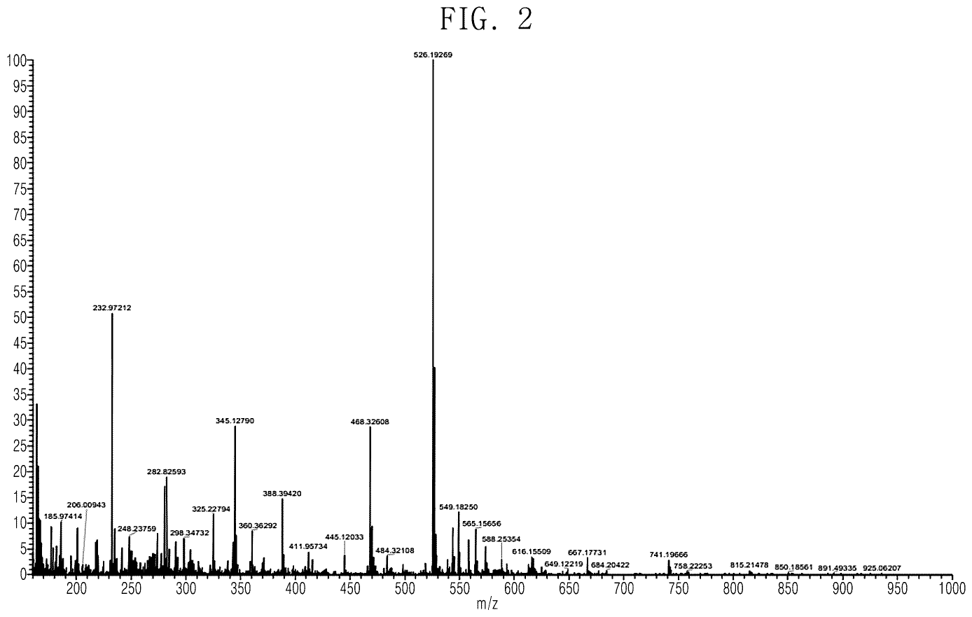

FIG. 1 is a view showing a ¹H NMR graph of HNF-propyne synthesized according to Example 1, and FIG. 2 is a view showing a mass spectrometry graph of HNF-propyne synthesized according to Example 1.

Referring to FIGS. 1 and 2, it is confirmed that HNF-propyne having a propargyl group containing a carbon-carbon triple bond was produced by reacting propargyl bromide with an —OH functional group of 9,9-bis(6-hydroxy-2-naphthyl)fluorene using K₂CO₃ as a base, according to Example 1 and Reaction Scheme 1.

### [Example 2] E-Beam Lithography Evaluation of HNF-Propyne (Formula 5-1)

An HNF-propyne solution (1 wt/vol %) in which HNF-propyne was dissolved in 2-methyl THF (tetrahydrofuran) was spin-coated onto a silicon substrate at 3,000 rpm for 60 seconds, and then heated at 50° C. for 1 minute to form a resist thin film (approximately 100 nm thick). Subsequently, the resist thin film was irradiated with an e-beam of 50 μC/cm² to 1,500 μC/cm² under an acceleration voltage of 80 keV, and then subjected to a development process performed using a mixed solvent of IPA and DIW (IPA: DI water=8:1) as a developer for 5 seconds to form a negative tone resist pattern.

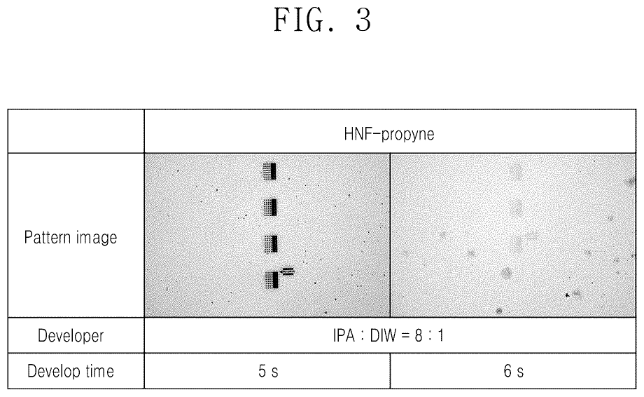

FIG. 3 is a scanning electron microscope image of a negative tone resist pattern formed by an e-beam lithography process according to Example 2.

Referring to FIG. 3, it is confirmed that a negative tone resist pattern is formed by performing e-beam lithography and development processes on a resist thin film formed using an HNF-propyne solution according to Example 2. Consequently, it is confirmed that the propargyl group containing a carbon-carbon triple bond may induce a decrease in the solubility of the resist thin film under e-beam irradiation. However, it is confirmed that the resist thin film is dissolved in the developer and removed as the development time of the development process increases.

### [Example 3] Synthesis of DHPNF (Core Structure of Formula 7)

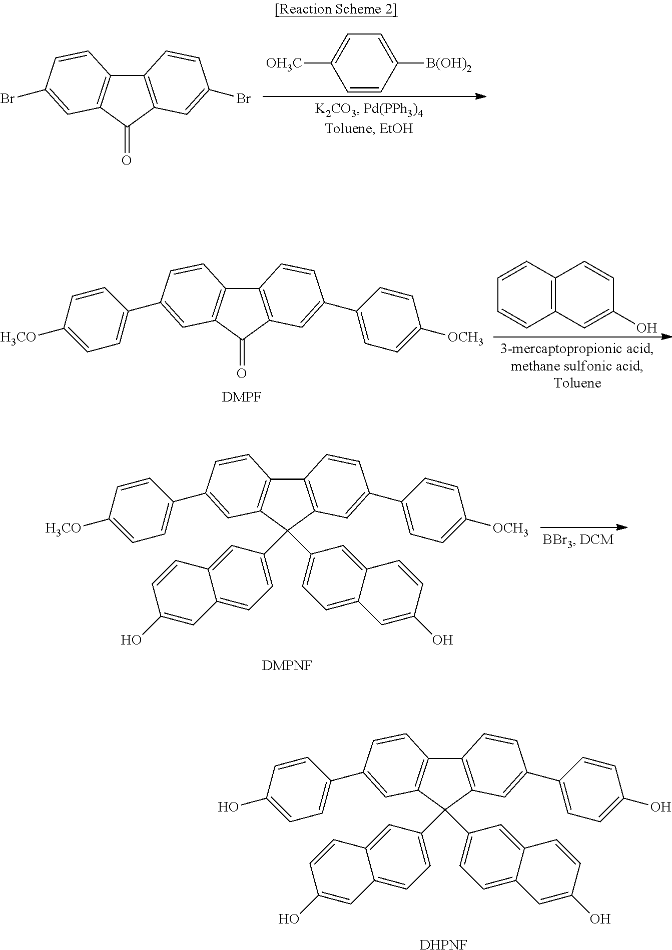

Synthesis of DMPF Introduced with a Methoxy Phenyl Unit:

2,7-dibromo-9-fluorenone (3.0 g, 8.88 mmol), 4-(methoxy)phenyl boronic acid (3.37 g, 22.19 mmol), 2 M K₂CO₃ aqueous solution (2.8 g, 19.97 mmol), and Pd(PPh₃)₄ (0.1 g, 0.09 mmol) were added to a 500 cm³ round-bottom flask, and toluene (100 cm³) and ethanol (25 cm³) were further added as solvents to form a reaction solution. The reaction solution was stirred at 80° C. for 12 hours to form a reactant. After the reaction was completed, water (100 cm³) was added to the reactant, and the mixture was then concentrated under reduced pressure. The concentrated reactant was diluted using dichloromethane (150 cm³), and an organic solvent layer of the reactant was washed twice with water, and then washed once more using a saturated aqueous sodium chloride solution (150 cm³). Subsequently, moisture contained in the organic solvent layer was removed using anhydrous MgSO₄ and the resulting organic solvent layer was concentrated under reduced pressure. The resulting product was dissolved in chloroform (20 cm³) and the solution was then allowed to stand at −20° C. to precipitate a solid. The precipitated solid was filtered through a filtration process under reduced pressure using cooled hexane to obtain DMPF (2.3 g, 66%) as a red solid.

¹H NMR (400 MHz, CDCl₃): δ=7.90 (s, Ar—H), 7.7 (d, J=8.0 Hz, Ar—H), 7.60-7.57 (m, Ar—H), 7.02 (d, J=9.0 Hz, Ar—H), 3.89 (s, 6H, —OCH₃)

Synthesis of DMPNF Introduced with a Naphthol Unit:

DMPF (2.3 g, 5.86 mmol), 2-naphthol (2.03 g, 14.07 mmol), 3-mercaptopropionic acid (0.06 g, 0.59 mmol), and methanesulfonic acid (2.25 g, 23.44 mmol) were added to a 100 cm³ round-bottom flask, and toluene (11.5 cm³) was further added as a solvent to form a reaction solution. The reaction solution was stirred at 50° C. for 12 hours to form a reactant. After the reaction was completed, the reactant was neutralized with a 2 M NaOH aqueous solution and diluted using ethyl acetate (150 cm³). Subsequently, an organic solvent layer of the reactant was washed twice with water, and then washed once more using a saturated aqueous sodium chloride solution (150 cm³). Moisture contained in the organic solvent layer was then removed using anhydrous MgSO₄, and the organic solvent layer, after moisture removal, was concentrated under reduced pressure. The resulting product was purified through column chromatography using a mixed solvent of dichloromethane and ethyl acetate (dichloromethane:ethyl acetate=15:1) as a mobile phase, and concentrated under reduced pressure. Consequently, DMPNF (2.7 g, 69%) was obtained as a red solid.

¹H NMR (400 MHz, acetone-d₆): δ=8.57 (s, 2H, —OH), 7.99 (d, J=8.1 Hz, 2H, Ar—H), 7.86 (d, J=1.3 Hz, 2H, Ar—H), 7.3 (d, J=1.9 Hz, 2H, Ar—H), 7.70-7.66 (m, 4H, Ar—H), 7.60 (d, J=9.1 Hz, 2H, Ar—H), 7.57 (dd, J=6.6, 1.9 Hz, 4H, Ar—H), 7.54 (dd, J=8.8, 1.9 Hz, 2H, Ar—H), 7.19 (d, J=2.5 Hz, 2H, Ar—H), 7.09 (dd, J=8.9, 2.4 Hz, 2H, Ar—H), 6.98-6.95 (m, 4H, Ar—H), 3.79 (s, 6H, —OCH₃)/m/z (ESI-TOF-MS) 662.24554 ([M+H]⁺. C₄₇H₃₄O₄H requires M, 663.78).

Synthesis of DHPNF with a Methoxy Unit Substituted by a Hydroxyl Functional Group:

DMPNF (2.7 g, 4.07 mmol) and N₂-bubbled dichloromethane (108 cm³) were added to a 500 cm³ round-bottom flask. Then, BBr₃ (1.0 M in dichloromethane) (6.12 g, 24.44 mmol) was further added at −78° C. and the mixture was stirred for 10 minutes to form a reaction solution. Subsequently, the reaction solution was stirred at room temperature for 6 hours to form a reactant. The reactant was diluted using water (100 cm³) and ethyl acetate (200 cm³). Thereafter, an organic solvent layer of the reactant was washed twice with water, and then washed once more using a saturated aqueous sodium chloride solution (150 cm³). Moisture contained in the organic solvent layer was then removed using anhydrous MgSO₄, and the organic solvent layer, after moisture removal, was concentrated under reduced pressure to obtain DHPNF (2.5 g, 97%) as a brown solid.

¹H NMR (400 MHz, DMSO-d₆): δ=9.67 (s, 2H, —OH), 9.51 (s, 2H, —OH), 7.99 (d, J=9.0 Hz, 2H, Ar—H), 7.69-7.58 (m, 8H, Ar—H), 7.54 (s, 2H, Ar—H), 7.45 (d, J=8.0 Hz, 4H, Ar—H), 7.38 (d, J=9.0 Hz, 2H, Ar—H), 7.06 (s, 2H, Ar—H), 7.69 (d, J=9.0 Hz, 2H, Ar—H), 6.81 (d, J=9.0 Hz, 4H, Ar—H)

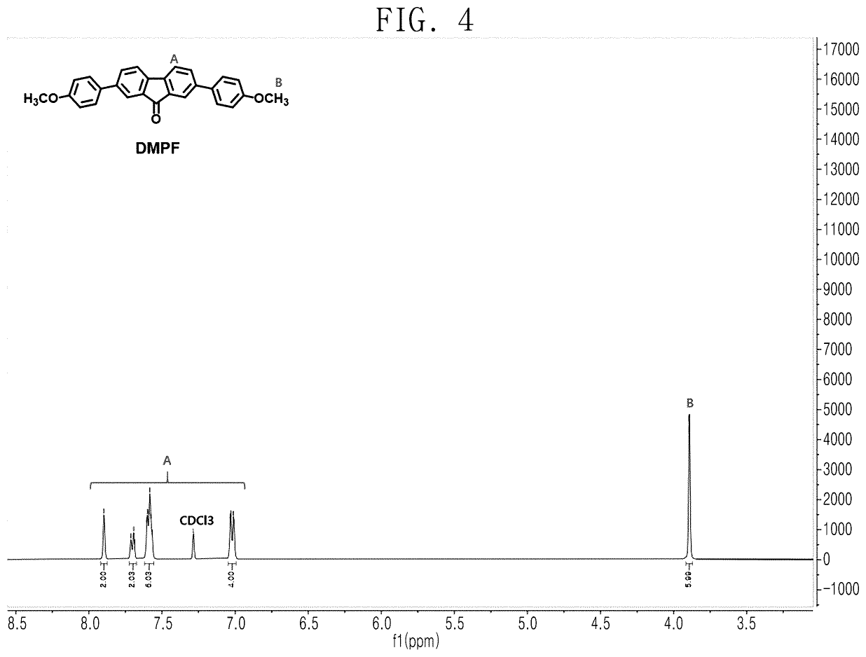

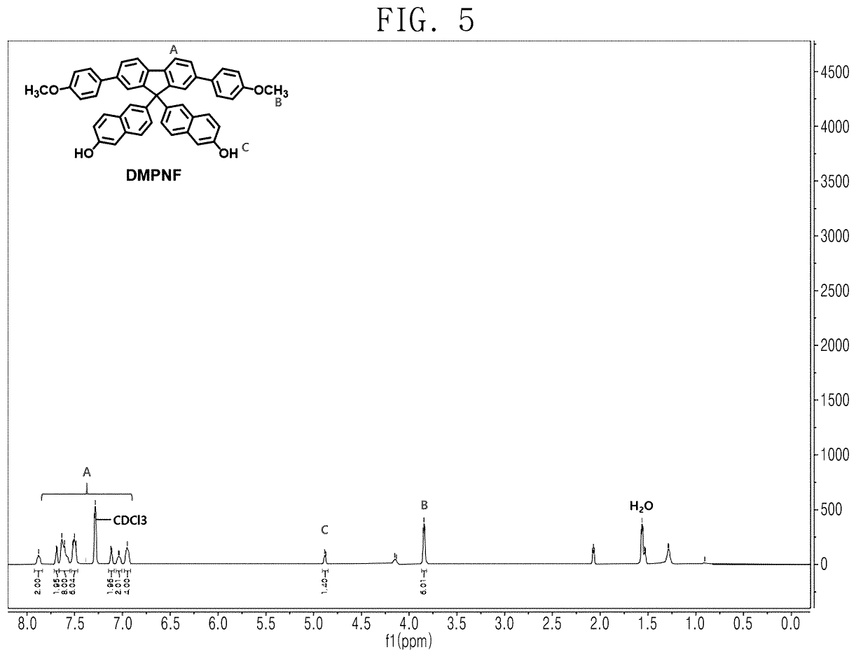

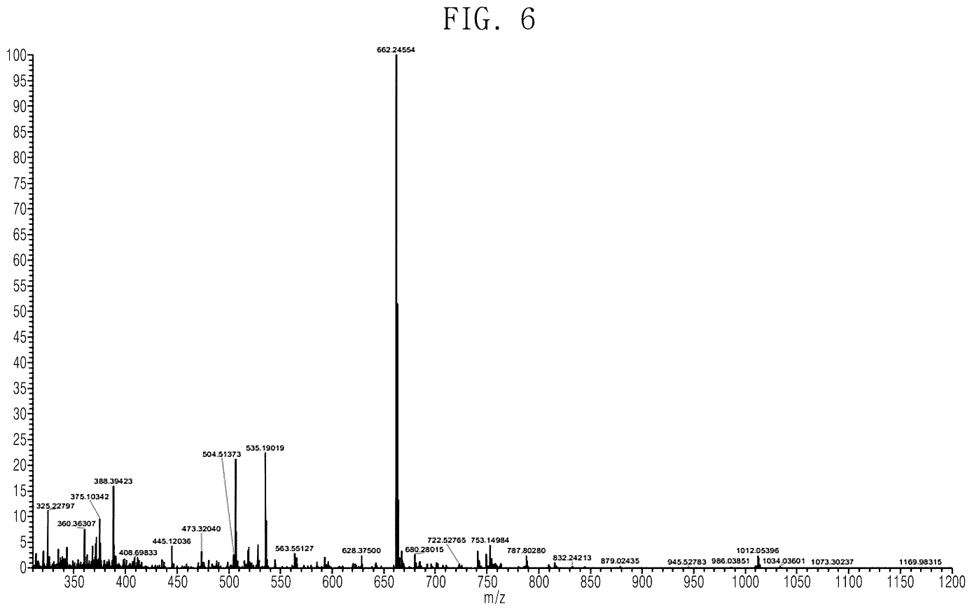

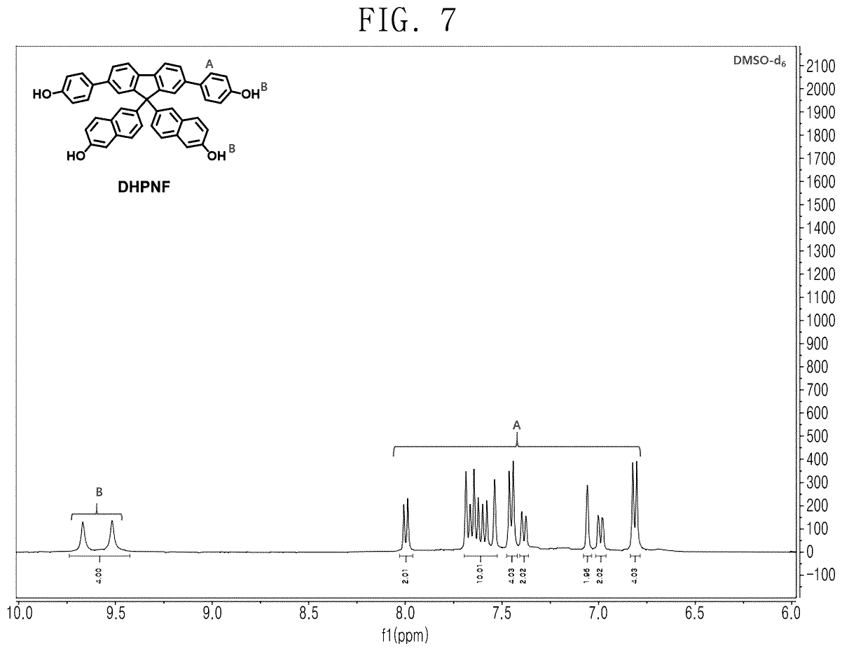

FIG. 4 is a view showing a ¹H NMR graph of DMPF synthesized according to Example 3. FIG. 5 is a view showing a ¹H NMR graph of DMPNF synthesized according to Example 3, and FIG. 6 is a view showing a mass spectrometry graph of DMPNF synthesized according to Example 3. FIG. 7 is a view showing a ¹H NMR graph of DHPNF synthesized according to Example 3.

Referring to FIGS. 4 to 7, according to Example 3 and Reaction Scheme 2, DHPNF was synthesized via 2,7-dibromo-9-fluorenone, DMPF, and DMPNF. In FIG. 4, it is confirmed that DMPF was produced by coupling 4-methoxy phenol with 2,7-dibromo-9-fluorenone. In FIGS. 5 and 6, it is confirmed that DMPNF introduced with a naphthol unit was produced through an electrophilic aromatic substitution reaction of DMPF and 2-naphthol. In FIG. 7, it is confirmed that DHPNF was produced by substituting the methoxy unit of DMPNF with an —OH group through a demethylation reaction using BBr₃.

### [Example 4] Synthesis of Bis-BPF (Core Structure of Formula 8) and Bis-BNF (Core Structure of Formula 9)

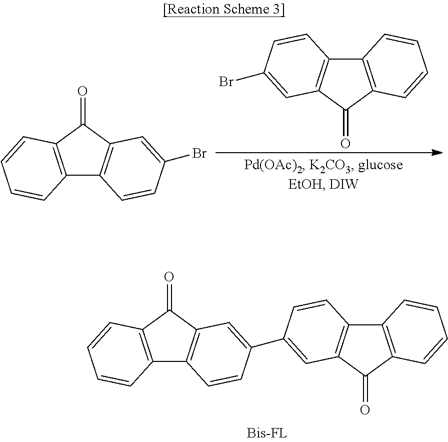 [SMILES: O=C1c2ccccc2-c2ccc(Br)cc21 ; O=C1c2ccccc2-c2ccc(Br)cc21 ; O=C1c2ccccc2-c2ccc(-c3ccc4c(c3)C(=O)c3ccccc3-4)cc21]

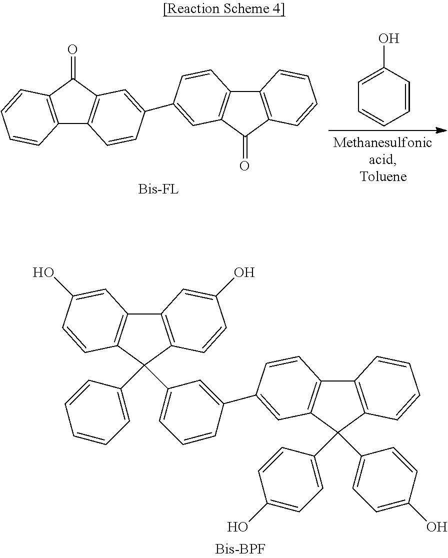

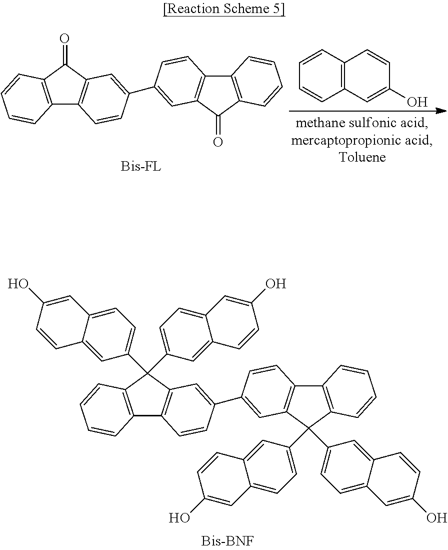

### Synthesis of Bis-FL (Reaction Scheme 3):

2-bromo-9-fluorenone (1.00 g, 3.86 mmol), Pd(OAc)₂ (43.3 mg, 0.19 mmol), glucose (0.70 g, 3.86 mmol), and K₂CO₃ (1.07 g, 7.7 mmol) diluted in DI water (10 cm³) were added to a 50 cm³ round-bottom flask, and ethanol (10 cm³) was further added as a solvent to form a reaction solution. The reaction solution was stirred at 80° C. for 12 hours to form a reactant. After the reaction was completed, the reactant was diluted with dichloromethane (100 cm³), and an organic solvent layer of the reactant was washed using water and a saturated aqueous NaCl solution. Moisture in the organic solvent layer was then removed using anhydrous MgSO₄, and the organic solvent layer, after moisture removal, was concentrated under reduced pressure to precipitate a solid product. The product was dispersed in dichloromethane (20 cm³) and filtered through a filtration process under reduced pressure to obtain Bis-FL (0.31 g, 44%) as a yellow solid.

¹H NMR (400 MHz, CDCl₃): δ=7.86 (dd, J=1.8, 0.6 Hz, 2H, Ar—H), 7.79 (dd, J=7.8, 1.8 Hz, 2H, Ar—H), 7.72 (dt, J=7.3, 1.0 Hz, 2H, Ar—H), 7.64 (dd, J=7.7, 0.6 Hz, 2H, Ar—H), 7.60 (dt, J=7.4, 1.0 Hz, 2H, Ar—H), 7.55 (td, J=7.4, 1.2 Hz, 2H, Ar—H), 7.35 (td, J=7.3, 1.2 Hz, 2H, Ar—H)

Synthesis of Bis-BPF Introduced with a Phenol Unit (Reaction Scheme 4):

Bis-FL (0.82 g, 2.3 mmol), phenol (3.3 g, 23 mmol), and methanesulfonic acid (2.2 g, 23 mmol) were added to a 100 cm³ round-bottom flask, and toluene (16 cm³) was further added as a solvent to form a reaction solution. The reaction solution was stirred at 50° C. for 12 hours to form a reactant. After the reaction was completed, the reactant was neutralized using a 2.0 M NaOH aqueous solution, and then the reactant was diluted using ethyl acetate (100 cm³). An organic solvent layer of the reactant was washed using water and a saturated NaCl aqueous solution, and moisture in the organic solvent layer was removed using anhydrous MgSO₄. The organic solvent layer, after moisture removal, was concentrated under reduced pressure to precipitate a solid product. The product was dispersed in ethyl acetate (20 cm³) and filtered through a filtration process under reduced pressure to obtain Bis-BPF (1.4 g, 87%) as a white solid.

¹H NMR (400 MHz, DMSO-d6): δ=9.29 (s, 4H, —OH), 7.95 (d, J=8.0 Hz, 2H, Ar—H), 7.93-7.88 (m, 2H, Ar—H), 7.63 (dd, J=8.0, 1.7 Hz, 2H, Ar—H), 7.52 (d, J=1.7 Hz, 2H, Ar—H), 7.38 (td, J=7.5, 1.2 Hz, 4H, Ar—H), 7.34-7.28 (m, 2H, Ar—H), 6.98-6.88 (m, 8H, Ar—H), 6.68-6.60 (m, 8H, Ar—H)

Synthesis of Bis-BNF Introduced with a Naphthol Unit (Reaction Scheme 5):

Bis-FL (0.66 g, 1.8 mmol), 2-naphthol (1.3 g, 9.2 mmol), methanesulfonic acid (1.4 g, 15 mmol), and 3-mercaptopropionic acid (0.04 g, 0.37 mmol) were added to a 100 cm³ round-bottom flask, and toluene (12 cm³) was further added as a solvent to form a reaction solution. The reaction solution was stirred at 50° C. for 12 hours to form a reactant. After the reaction was completed, the reactant was neutralized using a 2.0 M NaOH aqueous solution, and then the reactant was diluted using ethyl acetate (100 cm³). An organic solvent layer of the reactant was washed using water and a saturated aqueous NaCl solution. Moisture in the organic solvent layer was then removed using anhydrous MgSO₄, and the organic solvent layer, after moisture removal, was concentrated under reduced pressure to precipitate a solid product. The product was dispersed in ethyl acetate (20 cm³) and filtered through a filtration process under reduced pressure to obtain Bis-BNF (0.51 g, 31%) as a brown solid.

1H NMR (400 MHz, acetone-d6): δ=8.56 (s, 4H, —OH), 7.98 (d, J=8.0 Hz, 2H, Ar—H), 7.95 (d, J=7.5 Hz, 2H, Ar—H), 7.89-7.86 (m, 2H, Ar—H), 7.73 (dd, J=8.0, 1.7 Hz, 2H), 7.62-7.56 (m, 10H, Ar—H), 7.54 (d, J=8.9 Hz, 4H, Ar—H), 7.43 (td, J=7.5, 1.1 Hz, 2H, Ar—H), 7.38 (dd, J=8.8, 1.9 Hz, 4H Ar—H), 7.33 (td, J=7.5, 1.2 Hz, 2H, Ar—H), 7.16 (d, J=2.5 Hz, 4H, Ar—H), 7.07 (dd, J=8.8, 2.4 Hz, 4H, Ar—H)

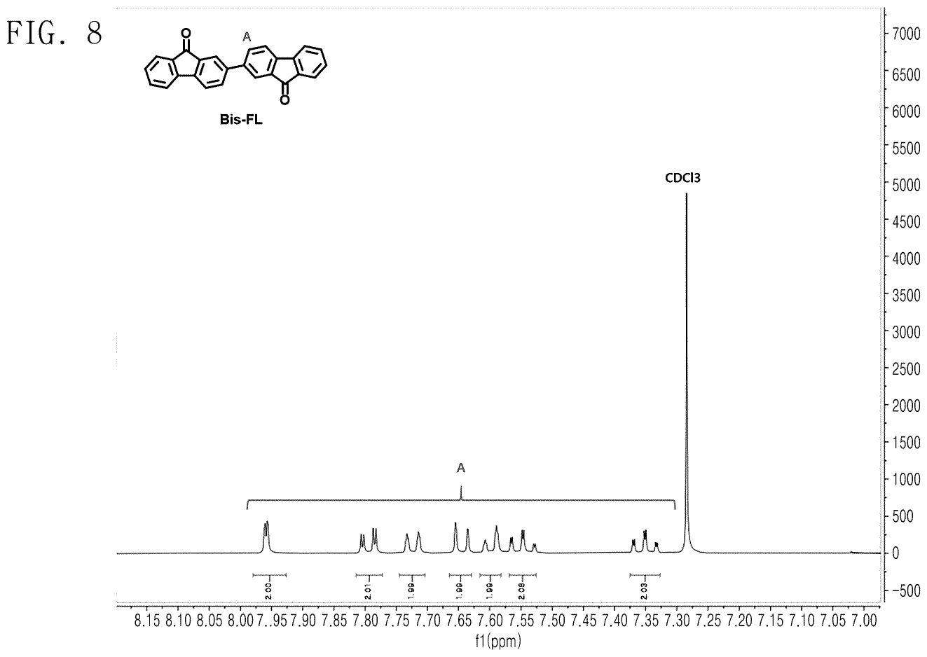

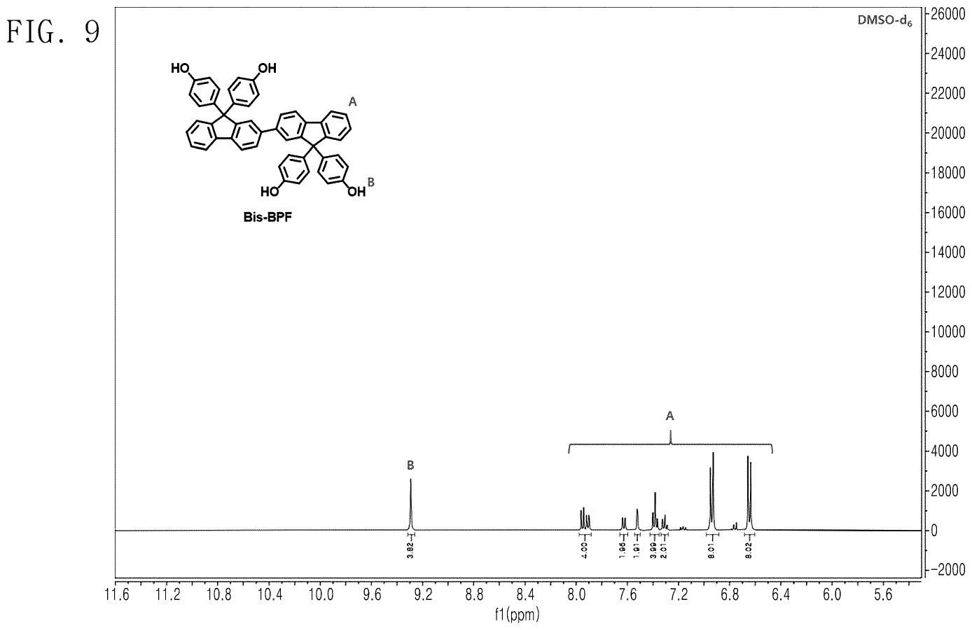

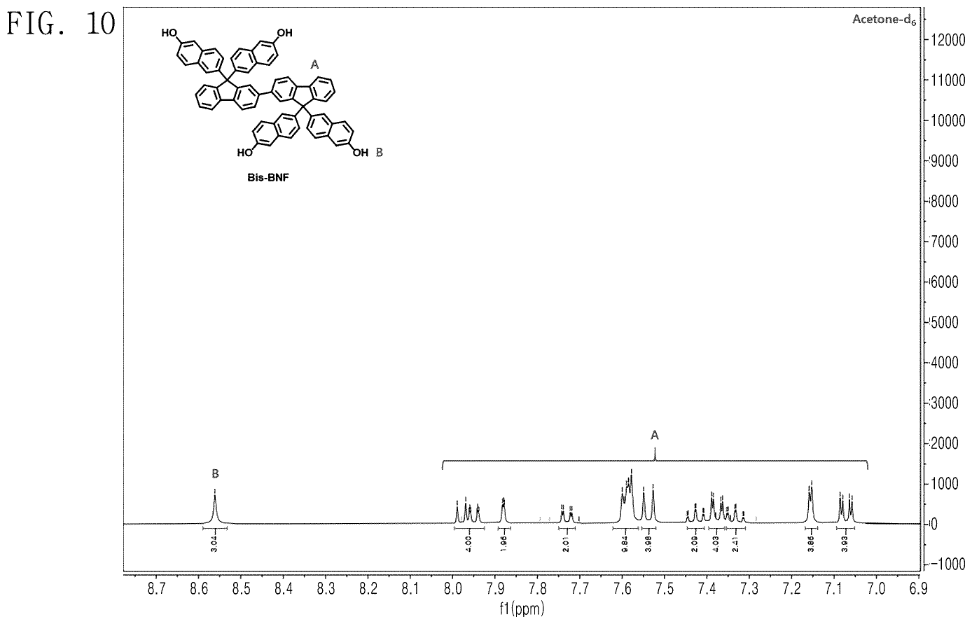

FIG. 8 is a view showing a ¹H NMR graph of Bis-FL synthesized according to Example 4, FIG. 9 is a view showing a ¹H NMR graph of Bis-BPF synthesized according to Example 4, and FIG. 10 is a view showing a ¹H NMR graph of Bis-BNF synthesized according to Example 4.

Referring to FIG. 8, it is confirmed that Bis-FL is synthesized through homo coupling of 2-bromo-9-fluorenone according to Example 4 and Reaction Scheme 3.

Referring to FIG. 9, it is confirmed that a phenol unit may be introduced into Bis-FL through an electrophilic aromatic substitution reaction according to Example 4 and Reaction Scheme 4, and thus, Bis-BPF is synthesized.

Referring to FIG. 10, it is confirmed that a naphthol unit may be introduced into Bis-FL through an electrophilic aromatic substitution reaction according to Example 4 and Reaction Scheme 5, and thus, Bis-BNF is synthesized.

### [Example 5] Thermogravimetric Analysis of HNF (Core Structure of Formula 5), DHPNF (Core Structure of Formula 7), and Bis-BNF (Core Structure of Formula 9

For each of HNF, DHPNF, and Bis-BNF, weight changes were determined by maintaining the temperature at 40° C. for 10 minutes in a nitrogen atmosphere and then increasing the temperature to 830° C. at a rate of 10° C./min, using thermal gravimetric analysis (TGA, TGA Q50, TA Instruments, USA).

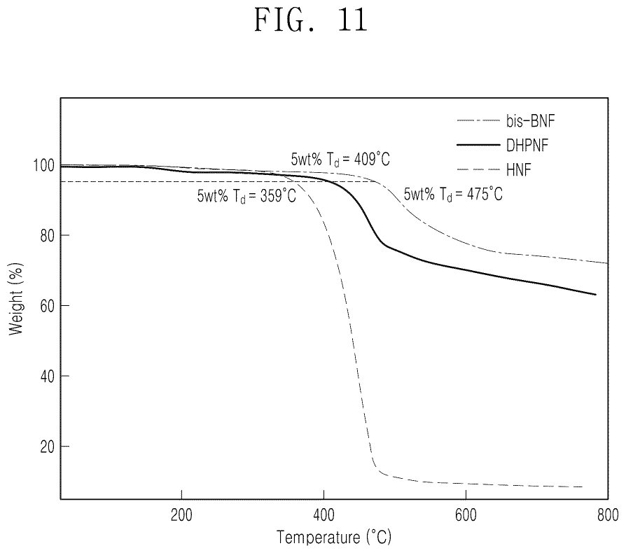

FIG. 11 is a view showing a thermogravimetric analysis graph of HNF, DHPNF, and Bis-BNF according to Example 5.

Referring to FIG. 11, it is confirmed that 5 wt % weight loss temperatures (5 wt % T_{d}) are 359° C. for HNF, 409° C. for DHPNF, and 475° C. for Bis-BNF. That is, it is confirmed that Bis-BNF has the most superior thermal stability.

### [Example 6] Differential Scanning Calorimetry Analysis of HNF (Core Structure of Formula 5), DHPNF (Core Structure of Formula 7), and Bis-BNF (Core Structure of Formula 9)

Glass transition temperatures were measured for HNF, DHPNF, and Bis-BNF, using a differential scanning calorimetry (DSC) device (DSC 200 F3, NETZSCH). The glass transition temperatures were measured using second heating data obtained during the process of heating and cooling each sample from −70° C. to 300° C. at a rate of 10° C./min in a nitrogen atmosphere.

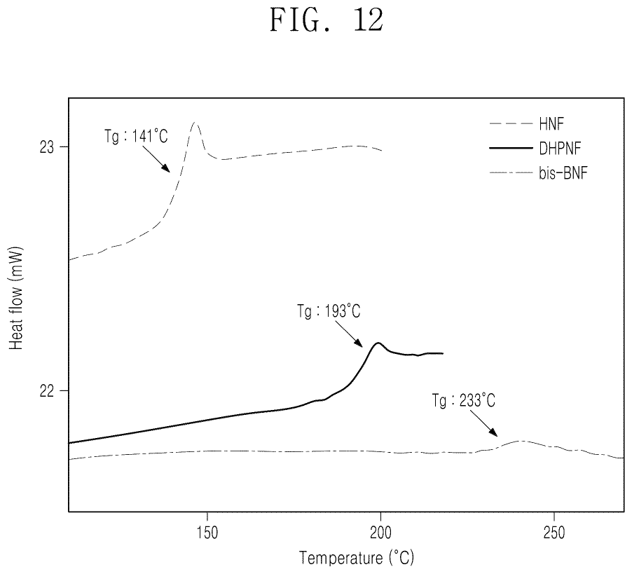

FIG. 12 is a view showing a graph of differential scanning calorimetry analysis of HNF, DHPNF, and Bis-BNF according to Example 6.

Referring to FIG. 12, the glass transition temperatures (Tg) were measured to be 141° C. for HNF, 193° C. for DHPNF, and 233° C. for Bis-BNF. Therefore, it is confirmed that Bis-BNF, with the highest glass transition temperature, has the most superior thermal stability.

### [Example 7] Etch Resistance Evaluation of HNF (Core Structure of Formula 5) and DHPNF (Core Structure of Formula 7)

To evaluate the etch resistance of resist compounds according to embodiments of the inventive concept, the etch rates of HNF and DHPNF were measured. As Comparative Example, the etch rate of a commercial KrF resist (DHK-BF511, Dongjin Semichem, South Korea) was measured.

First, a HNF solution (6 wt/vol %) in which HNF was dissolved in 2-methyl THF was spin-coated on a silicon substrate at 1500 rpm for 60 seconds and heated at 60° C. for 1 minute to form a first resist thin film having a thickness of approximately 435 nm.

In addition, a DHPNF solution (6 wt/vol %) in which DHPNF was dissolved in 2-methyl THF was spin-coated on a silicon substrate at 1,500 rpm for 60 seconds and heated at 60° C. for 1 minute to form a second resist thin film having a thickness of approximately 435 nm.

As Comparative Example, a commercial KrF resist solution (DHK-BF511, Dongjin Semichem, South Korea) was spin-coated on a silicon substrate at 1,500 rpm for 60 seconds and heated at 80° C. for 1 minute to form a third resist thin film having a thickness of approximately 577 nm.

Dry etching was performed using a reactive ion etching mode of the etching equipment under conditions of 100 W, 100 kHz, O₂ (4 sccm), and CF₄ (4 sccm). The residual film thickness was measured according to etching times (0, 10, 20, 30, 40, 50, and 60 seconds) using an Alpha-step® D-300 stylus profiler manufactured by Kla-Tencor. As a result, under the etching conditions described above, the etch rates of the first resist film (HNF), the second resist film (DHPNF), and the third resist film (commercial KrF resist) were measured to be 2.95 nm/see, 2.58 nm/see, and 3.29 nm/see, respectively.

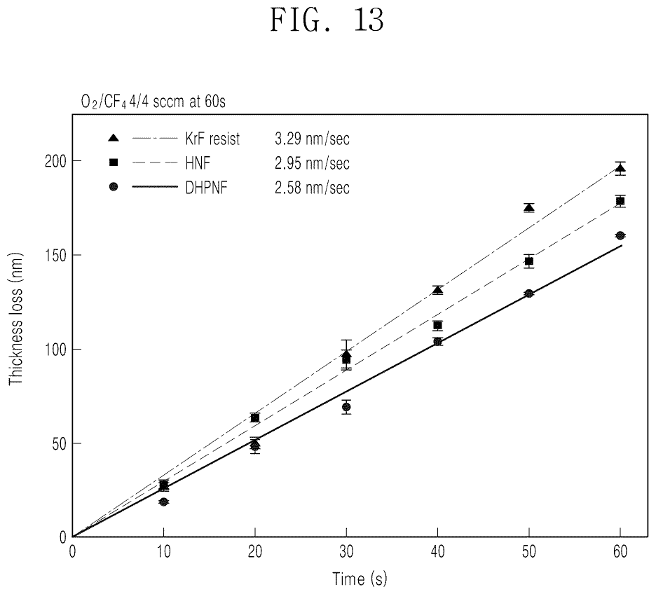

FIG. 13 is a graph showing the results of measuring dry etch rates of HNF and DHPNF according to Example 7.

Referring to FIG. 13, the etch rate of the commercial KrF resist thin film was measured to be 3.29 nm/see, the etch rate of the resist thin film formed using HNF was measured to be 2.95 nm/see, and the etch rate of the resist thin film formed using DHPNF was measured to be 2.58 nm/sec. Accordingly, it is confirmed that HNF and DHPNF, the resist core structures, exhibit superior etch resistance compared to the KrF resist thin film. Furthermore, it is confirmed that DHPNF, which has a core structure containing a relatively larger number of benzene rings, exhibits superior etch resistance compared to HNF.

### [Example 8] Synthesis of DHPNF-Propyne (Formula 7-1) Introduced with a Carbon-Carbon Triple Bond

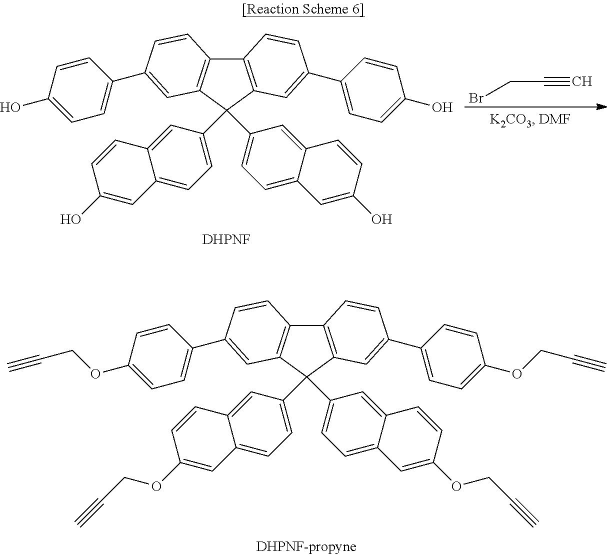

DHPNF (0.618 g, 0.97 mmol), propargyl bromide (0.70, 5.85 mmol), and K₂CO₃ (0.81 g, 5.85 mmol) were added to a 250 cm³ round-bottom flask, and DMF (5 cm³) was further added as a solvent to form a reaction solution. The reaction solution was stirred at room temperature for 12 hours to form a reactant. After the reaction was completed, the reactant was diluted using ethyl acetate (100 cm³), and an organic solvent layer of the reactant was washed using water and a saturated aqueous NaCl solution. Subsequently, moisture in the organic solvent layer was removed using anhydrous MgSO₄. The organic solvent layer, after moisture removal, was concentrated under reduced pressure to form a product. The product was purified through column chromatography using a mixed solvent of ethyl acetate and hexane (ethyl acetate:hexane=1:3) as a mobile phase, and then concentrated under reduced pressure. Consequently, DHPNF-propyne (0.464 g, 61%) was obtained as a light brown solid.

¹H NMR (400 MHz, CDCl₃): δ=7.88 (d, J=8.0 Hz, 2H, Ar—H), 7.71-7.59 (m, 10H, Ar—H), 7.51 (d, J=8.7 Hz, 6H, Ar—H), 7.22 (d, J=2.8 Hz, 2H, Ar—H), 7.13 (dd, J=9.0, 2.8 Hz, 2H, Ar—H), 7.02 (m, 4H, Ar—H), 4.79 (d, J=2.2 Hz, 4H, —OCH₂—), 4.72 (d, J=2.3 Hz, 4H, —OCH₂—), 2.54-2.52 (m, 4H, —C≡CH)

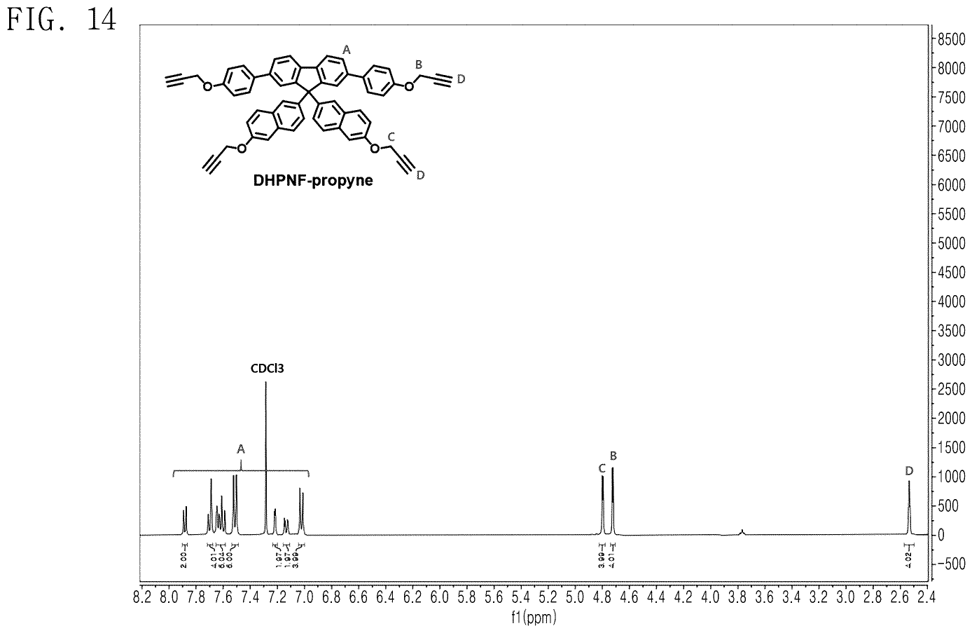

FIG. 14 is a view showing a ¹H NMR graph of DHPNF-propyne synthesized according to Example 8.

Referring to FIG. 14, it is confirmed that DHPNF-propyne having a propargyl group containing a carbon-carbon triple bond was produced through an S_{N}2 reaction between four hydroxy groups of DHPNF and propargyl bromide according to Example 8 and Reaction Scheme 6.

### [Example 9] E-Beam Lithography Evaluation of DHPNF-Propyne (Formula 7-1)

A solution of 1,3-divinyl-1,1,3,3-tetramethyldisilazane (0.25 vol/vol %) dissolved in propylene glycol monomethyl ether acetate (PGMEA) was spin-coated on a silicon substrate at 3,000 rpm for 30 seconds, and then heated at 130° C. for 1 minute to form an underlayer (e.g., a self-assembled monolayer). Then, a DHPNF-propyne solution (2 wt/vol %) dissolved in propylene glycol monomethyl ether (PGME) was spin-coated on the underlayer at 1,500 rpm for 60 seconds, and then heated at 100° C. for 1 minute to form a resist thin film (approximately 100 nm thick). Subsequently, an e-beam lithography process with different conditions was performed on the resist thin film as follows.

### Condition 1:

The resist thin film was irradiated with e-beams ranging from 50 C/cm² to 1,500 μC/cm² under an acceleration voltage of 80 keV. A development process was then performed for 30 seconds using a developer (a mixed solvent of isopropyl alcohol (IPA) and methyl isobutyl ketone (MIBK) at a ratio of 3:1) to form a negative tone resist pattern.

### Condition 2:

The resist thin film was irradiated with e-beams ranging from 1150 μC/cm² to 2150 μC/cm² under an acceleration voltage of 80 keV. A development process was then performed using a developer (1,3-bis(trifluoromethyl)benzene) for 40 seconds to form a negative tone resist pattern.

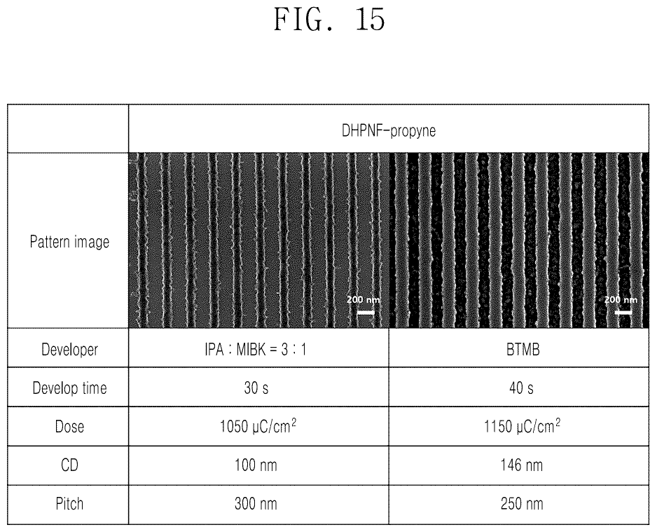

FIG. 15 is a scanning electron microscope image of a negative tone resist pattern formed by an e-beam lithography process according to Example 9.

Referring to FIG. 15, it is confirmed that a negative tone resist pattern is formed by performing e-beam lithography and development processes on a resist thin film formed using a DHPNF-propyne solution according to Example 9. The development process may use a mixed solvent of IPA and methyl isobutyl ketone (MIBK) (IPA:MIBK=3:1) or (1,3-bis(trifluoromethyl)benzene (BTMB) as a developer.

### [Example 10] Synthesis of DHPNF-GPE (Formula 7-2) Introduced with Both a Carbon-Carbon Triple Bond and a Hydroxy Functional Group

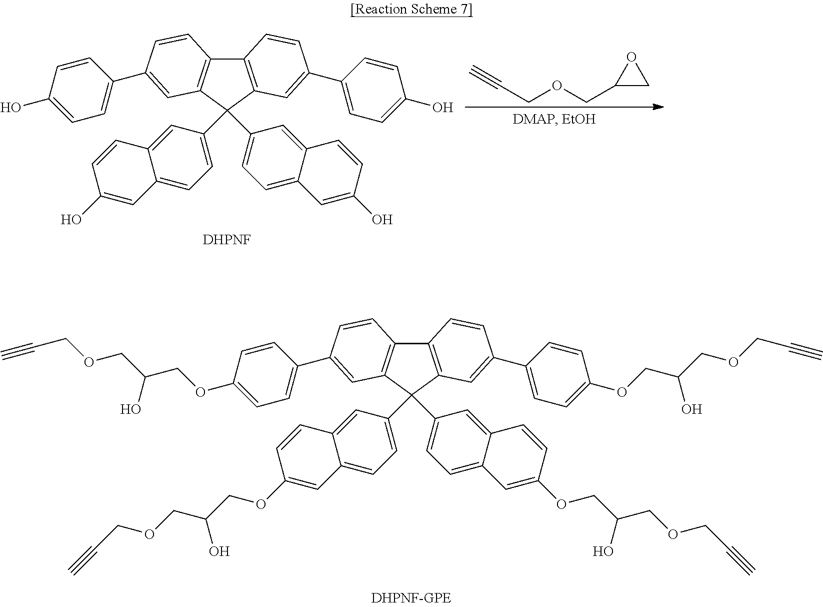

DHPNF (0.30 g, 0.47 mmol) and glycidyl propargyl ether (0.42 g, 3.8 mmol) were added to a 25 cm³ pressure reaction vessel (seal-tube), and 4-dimethylaminopyridine (DMAP, 11.5 mg, 0.09 mmol) and ethanol (4 cm³) were further added and sealed to form a reaction solution. The reaction solution was stirred at 90° C. for 12 hours to form a reactant. After the reaction was completed, the reactant was diluted using ethyl acetate (100 cm³). An organic solvent layer of the reactant was washed using water and a saturated aqueous NaCl solution. The organic solvent layer was dried using anhydrous MgSO₄, and the dried organic solvent layer was concentrated under reduced pressure to form a product. The product was purified through column chromatography using ethyl acetate as a mobile phase, and then concentrated again under reduced pressure to obtain DHPNF-GPE (0.21 g, 41%) as a yellow solid.

¹H NMR (400 MHz, CDCl₃): δ=7.88 (d, J=7.9 Hz, 2H, Ar—H) 7.72-7.54 (m, 12H, Ar—H), 7.50 (dd, J=7.5, 4.9 Hz, 4H, Ar—H), 7.17-7.07 (m, 4H, Ar—H), 4.27, 4.19 (m, 4H, —Ar—O—CH₂CH—), 4.22 (dd, J=3.5, 2.4 Hz, 8H, Ar—O—CH₂—), 4.15-4.00 (m, 8H, —OCH₂—C≡CH), 3.79-3.65 (m, 8H, —CH₂—O—CH₂—C≡CH), 2.44 (t, J=2.4 Hz, 4H, —C≡C—H)

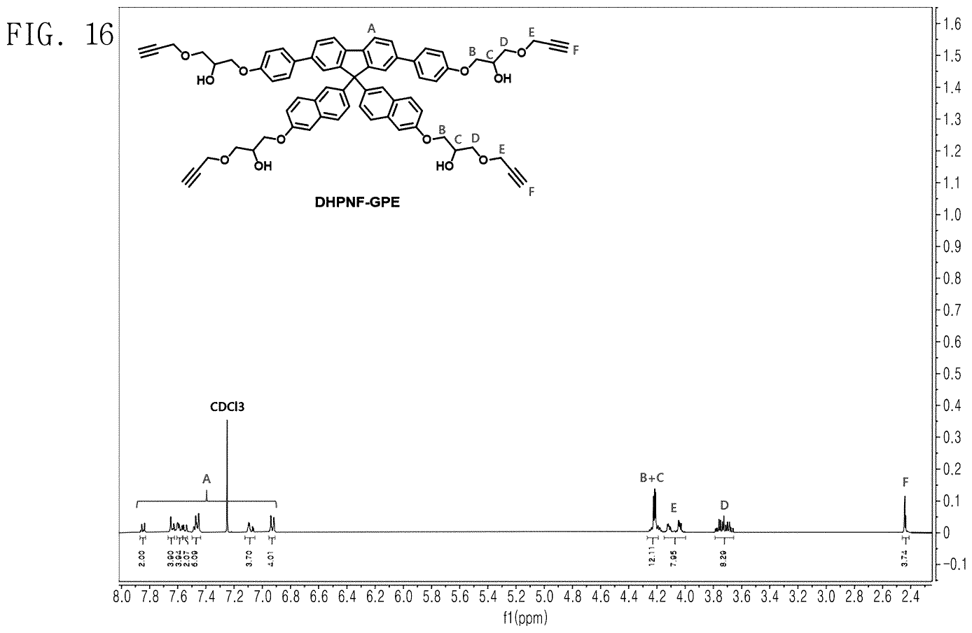

FIG. 16 is a view showing a ¹H NMR graph of DHPNF-GPE synthesized according to Example 10.

Referring to FIG. 16, it is confirmed that DHPNF-GPE having four carbon-carbon triple bonds and four hydroxyl groups was produced using a ring-opening reaction between DHPNF and glycidyl propargyl ether (GPE) according to Example 10 and Reaction Scheme 7.

### [Example 11] Synthesis of DHPNF-GAE (Formula 7-3) Introduced with Both a Carbon-Carbon Double Bond and a Hydroxy Functional Group

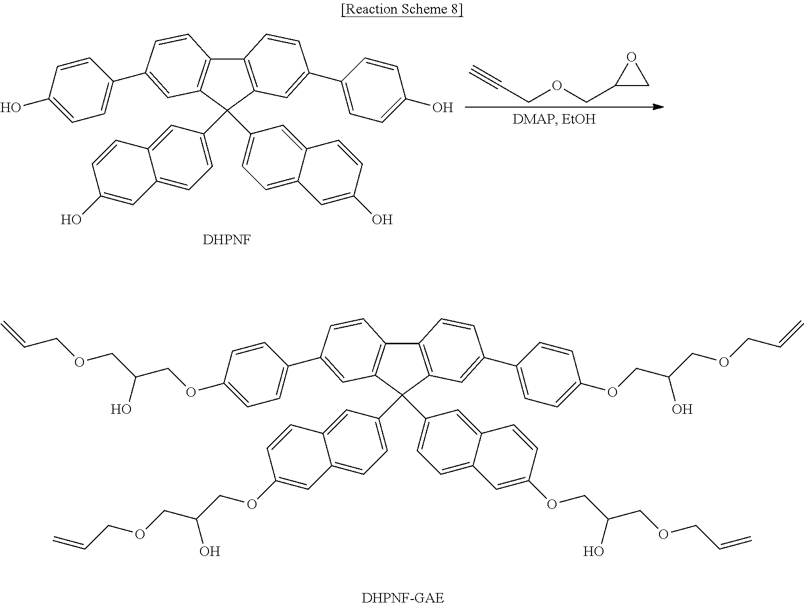

DHPNF (0.30 g, 0.47 mmol) and glycidyl allyl ether (0.43 g, 3.8 mmol) were added to a 25 cm³ seal-tube, and DMAP (11.5 mg, 0.09 mmol) and ethanol (4 cm³) were further added and sealed to form a reaction solution. The reaction solution was stirred at 90° C. for 12 hours to form a reactant. After the reaction was completed, the reactant was diluted using ethyl acetate (100 cm³). An organic solvent layer of the reactant was washed using water and a saturated aqueous NaCl solution. The organic solvent layer was dried using anhydrous MgSO₄, and the dried organic solvent layer was concentrated under reduced pressure to form a product. The product was purified through column chromatography using ethyl acetate as a solvent, and then concentrated again under reduced pressure to obtain DHPNF-GAE (0.29 g, 55%) as a yellow solid.

¹H NMR (400 MHz, CDCl₃): δ=7.88 (d, J=7.9 Hz, 2H, Ar—H) 7.72-7.54 (m, 12H, Ar—H), 7.50 (dd, J=7.5, 4.9 Hz, 4H, Ar—H), 7.17-7.07 (m, 4H, Ar—H), 6.96 (d, J=8.8 Hz, 4H, Ar—H), 6.01-5.83 (m, 4H, —CH₂CH═CH₂), 5.31 (dq, J=17.2, 1.7 Hz, 4H, —CH═CH₂), 5.22 (dq, J=10.5, 1.4 Hz, 4H, —CH═CH₂), 4.26-4.19 (m, 4H, HO—CH—), 4.19-4.09 (m, 8H, Ar—O—CH₂—), 4.09-4.06 (m, 8H, —O—CH₂—CHCH₂), 3.65 (ddt, J=14.8, 9.8, 4.5 Hz, 8H, —CH₂—O—CH₂—CH═CH₂)

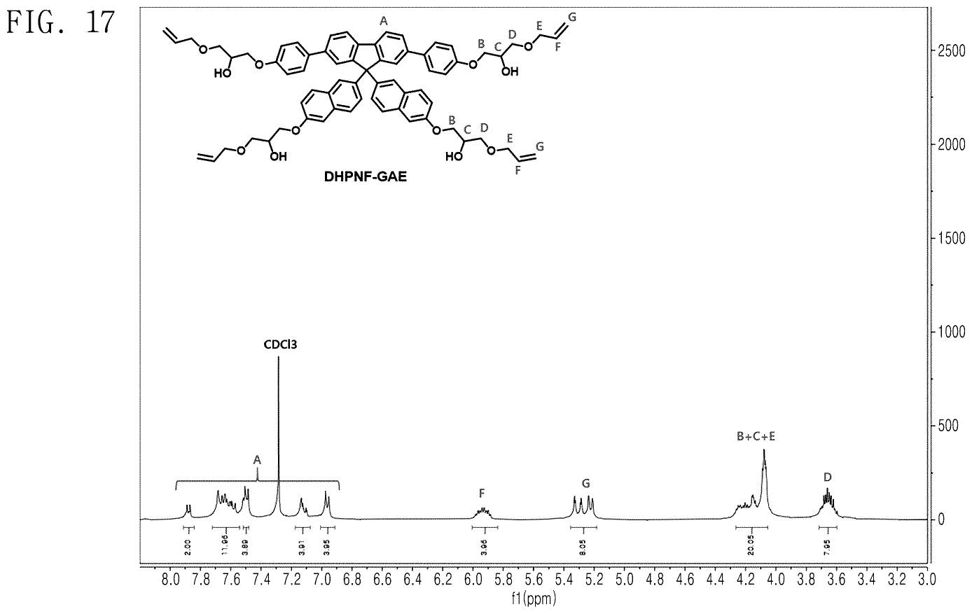

FIG. 17 is a view showing a ¹H NMR graph of DHPNF-GAE synthesized according to Example 11.

Referring to FIG. 17, it is confirmed that DHPNF-GAE having four carbon-carbon double bonds and four hydroxyl groups was produced using a ring-opening reaction between DHPNF and glycidyl allyl ether (GAE) according to Example 11 and Reaction Scheme 8.

### [Example 12] E-Beam Lithography Evaluation of DHPNF-GPE (Formula 7-2)

A solution of 1,3-divinyl-1,1,3,3-tetramethyldisilazane (0.25 vol/vol %) dissolved in PGMEA was spin-coated on a silicon substrate at 3,000 rpm for 30 seconds, and then heated at 130° C. for 1 minute to form an underlayer. Then, a DHPNF-GPE solution (2.2 wt/vol %) dissolved in MIBK was spin-coated on the underlayer at 2,000 rpm for 60 seconds, and then heated at 80° C. for 1 minute to form a resist thin film (approximately 100 nm thick). The resist thin film was irradiated with e-beams ranging from 500 μC/cm² to 1,100 μC/cm² under an acceleration voltage of 80 keV. Subsequently, a development process using a developer (toluene) was performed for 60 seconds to form a negative tone resist pattern.

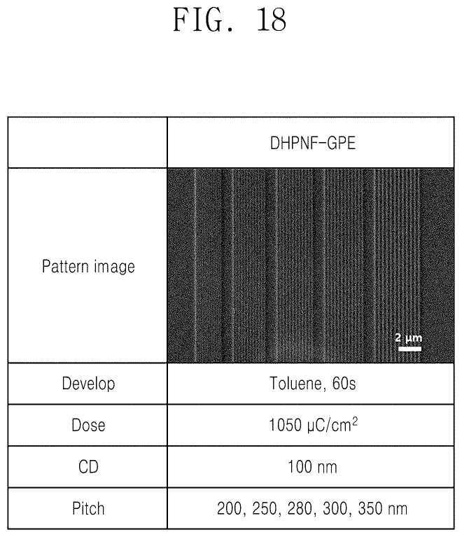

FIG. 18 is a scanning electron microscope image of a negative tone resist pattern formed by an e-beam lithography process according to Example 12.

Referring to FIG. 18, it is confirmed that a negative tone resist pattern is formed by performing e-beam lithography and development processes on a resist thin film formed using a DIPNF-GPE solution according to Example 12. The development process may use toluene as a developer.

### [Example 13] Evaluation of Changes in Solubility of DHPNF-GPE (Formula 7-2) and DHPNF-GAE (Formula 7-3) with Respect to Deep UV

First, a solution of 1,3-divinyl-1,1,3,3-tetramethyldisilazane (0.25 vol/vol %) dissolved in PGMEA was spin-coated on a silicon substrate at 3,000 rpm for 30 seconds, and then heated at 130° C. for 1 minute to form an underlayer.

### Evaluation of DHPNF-GPE (Formula 7-2) Resist Thin Film:

A DHPNF-GPE solution (2.2 wt/vol %) dissolved in MIBK was spin-coated on the underlayer at 2,000 rpm for 60 seconds, and then heated at 80° C. for 1 minute to form a first resist thin film (approximately 100 nm thick). The first resist thin film was irradiated with UV light having an energy range of 5 mJ/cm² to 250 mJ/cm² using a 254 nm UV exposure device. A development process using a developer (toluene) was performed for 60 seconds to form a negative tone resist pattern. By measuring the thickness of the resist pattern using an Alpha-step® D-300 stylus profiler manufactured by Kla-Tencor, a solubility change curve (contrast curve) according to the 254 nm UV irradiation dose was obtained.

### Evaluation of DHPNF-GAE (Formula 7-3) Resist Thin Film:

A DHPNF-GAE solution (2.2 wt/vol %) dissolved in MIBK was spin-coated on the underlayer at 2,000 rpm for 60 seconds, and then heated at 80° C. for 1 minute to form a second resist thin film (approximately 100 nm thick). The second resist thin film was irradiated with UV light having an energy range of 5 mJ/cm² to 250 mJ/cm² using a 254 nm UV exposure device. A development process using a developer (toluene) was performed for 60 seconds to form a negative tone resist pattern. By measuring the thickness of the resist pattern using an Alpha-step® D-300 stylus profiler manufactured by Kla-Tencor, a solubility change curve (contrast curve) according to the 254 nm UV irradiation dose was obtained.

FIG. 19 is a graph showing the results of evaluating solubility of a resist thin film for a deep UV lithography process according to Example 13.

Referring to FIG. 19, in the case of the DHPNF-GPE (Formula 7-2) resist thin film, it is confirmed that the thickness of the resist pattern is maintained at approximately 50% of the thickness of the initially coated resist thin film upon irradiation with a deep UV of 44.5 mJ/cm². In the case of the DHPNF-GAE (Formula 7-3) resist thin film, it is confirmed that the thickness of the resist pattern is maintained at approximately 50% of the thickness of the initially coated resist thin film upon irradiation with a deep UV of 112.1 mJ/cm². Consequently, it is confirmed that DHPNF-GPE (Formula 7-2) introduced with a carbon-carbon triple bond has superior sensitivity characteristics compared to DHPNF-GAE (Formula 7-3) introduced with a carbon-carbon double bond.

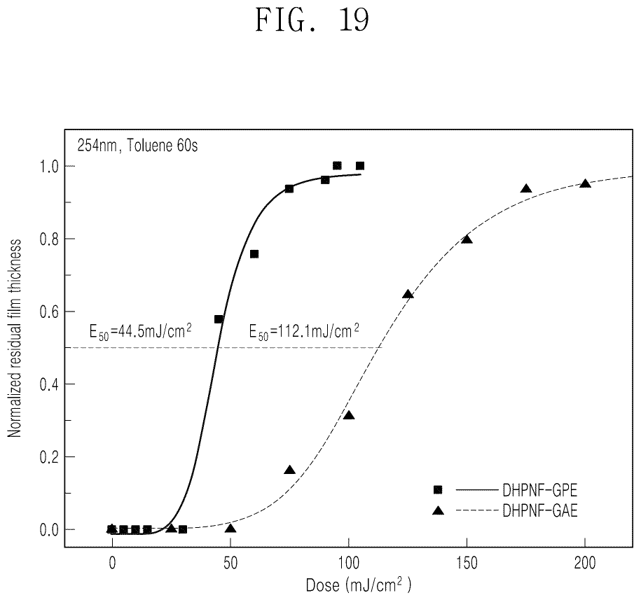

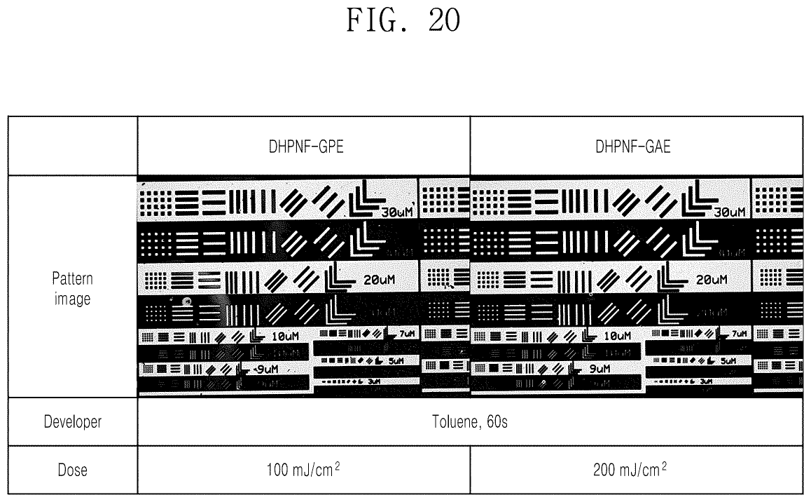

FIG. 20 is an optical microscope image of a negative tone resist pattern formed by a deep UV lithography process according to Example 13.

Referring to FIG. 20, it is confirmed that a negative tone resist pattern is formed by performing a deep UV lithography process at an exposure dose of 100 mJ/cm² on the DHPNF-GPE (Formula 7-2) resist thin film. It is confirmed that a negative tone resist pattern is formed by performing a deep UV lithography process at an exposure dose of 200 mJ/cm² on the DHPNF-GAE (Formula 7-3) resist thin film.

### [Example 14] Formation of a Cross-Linked Poly(4-Hydroxystyrene) (PHS) Underlayer

Poly(4-hydroxystyrene) (PHS, 1 wt/vol %), 1,3,4,6-tetrakis(methoxymethyl)glycoluril (TMMGU, 10 wt/wt % relative to PHS) as a cross-linking agent, and pyridinium p-toluenesulfonate (PPTS, 3 wt/wt % relative to PHS) as a thermal initiator were added to PGME to prepare an underlayer coating solution. The underlayer coating solution was spin-coated on a silicon substrate at 4,000 rpm for 60 seconds, and then heated at 150° C. for 5 minutes to form a cross-linked PHS underlayer thin film (approximately 15 nm thick).

### [Example 15] Evaluation of Developer of DHPNF-GPE (Formula 7-2) with/without a Cross-Linked PHS Underlayer

Evaluation 1: Case without a Cross-Linked PHS Underlayer

A DHPNF-GPE solution (2.2 wt/vol %) dissolved in MIBK was spin-coated on a silicon substrate at 2,000 rpm for 60 seconds, and then heated at 80° C. for 1 min to form a resist thin film (approximately 100 nm thick). The resist thin film was irradiated with UV light having an energy range of 50 mJ/cm² to 150 mJ/cm² using a 254 nm UV exposure device. Subsequently, a development process using toluene or o-xylene as a developer was performed for 30 seconds to form a negative tone resist pattern.

Evaluation 2: Case with a Cross-Linked PHS Underlayer

A cross-linked PHS underlayer was formed on a silicon substrate according to Example 14. A DHPNF-GPE solution (2.2 wt/vol %) dissolved in MIBK was spin-coated on the underlayer at 2,000 rpm for 60 seconds, and then heated at 80° C. for 1 minute to form a resist thin film (approximately 100 nm thick). The resist thin film was irradiated with UV light having an energy range of 400 mJ/cm² to 600 mJ/cm² using a 254 nm UV exposure device. Subsequently, a development process using heptanone, MIBK, or nBA as a developer was performed for 30 seconds to form a negative tone resist pattern.

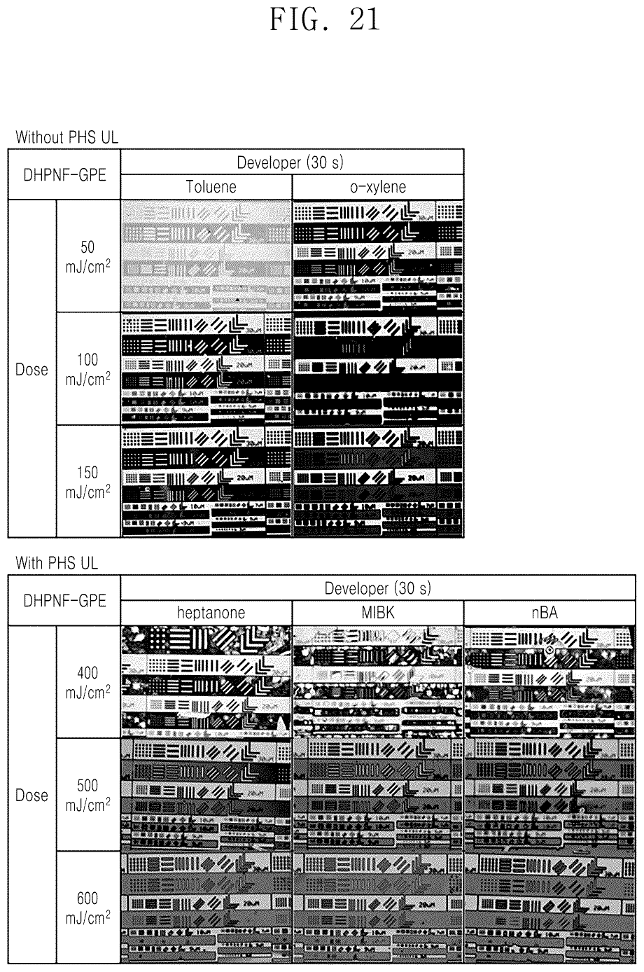

FIG. 21 is an optical microscope image of a negative tone resist pattern formed according to Example 15.

Referring to FIG. 21, it is confirmed that, in the case without a cross-linked PHS underlayer, only benzene-based organic solvents, such as toluene and o-xylene, may be used as developers. It is confirmed that, in the case with a cross-linked PHS underlayer, ketone and ester-based organic solvents, such as 2-heptanone, n-butyl acetate (nBA), and MIBK, may be used as developers.

### [Example 16] E-Beam Lithography Evaluation of DHPNF-GPE (Formula 7-2) on a Cross-Linked PHS Underlayer

A cross-linked PHS underlayer was formed on a silicon substrate according to Example 14. A DHPNF-GPE solution (2.2 wt/vol %) dissolved in MIBK was spin-coated on the underlayer at 2,000 rpm for 60 seconds, and then heated at 80° C. for 1 minute to form a resist thin film (approximately 100 nm thick). Then, the resist thin film was irradiated with e-beams ranging from 100 μC/cm² to 1,500 μC/cm² under an acceleration voltage of 80 keV. Subsequently, a development process using heptanone, MIBK, or nBA as a developer was performed to form a negative tone resist pattern.

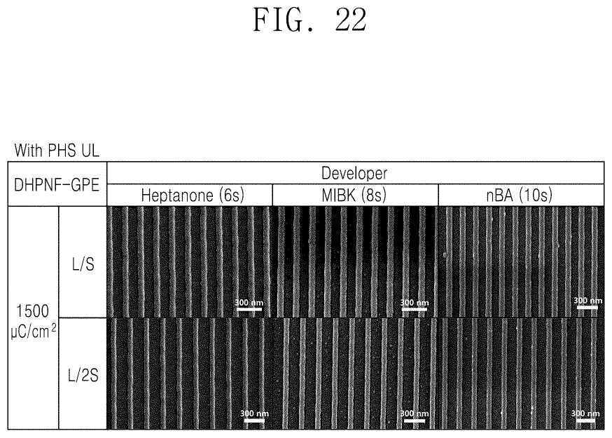

FIG. 22 is a scanning electron microscope image of a negative tone resist pattern formed by an e-beam lithography process according to Example 16.

Referring to FIG. 22, a DHPNF-GPE resist thin film was formed on the cross-linked PHS underlayer, and an e-beam lithography process was performed on the resist thin film according to Example 16. Thereafter, a development process using heptanone, MIBK, and nBA as developers was performed for 6, 8, and 10 seconds, respectively. Accordingly, it is confirmed that a negative tone resist pattern having a line width of 100 nm is formed.

### [Example 17] Thermogravimetric Analysis of HNF-Propyne (Formula 5-1), DHPNF-Propyne (Formula 7-1), DHPNF-GPE (Formula 7-2), and DHPNF-GAE (Formula 7-3)

To evaluate the thermal stability of resist compounds, thermogravimetric analysis was performed on HNF-propyne, DHPNF-propyne, DHPNF-GPE, and DHPNF-GAE. For each of HNF-propyne, DHPNF-propyne, DHPNF-GPE, and DHPNF-GAE, weight changes were determined by maintaining the temperature at 40° C. for 10 minutes in a nitrogen atmosphere and then increasing the temperature to 830° C. at a rate of 10° C./min, using thermal gravimetric analysis (TGA, TGA Q50, TA Instruments, USA).

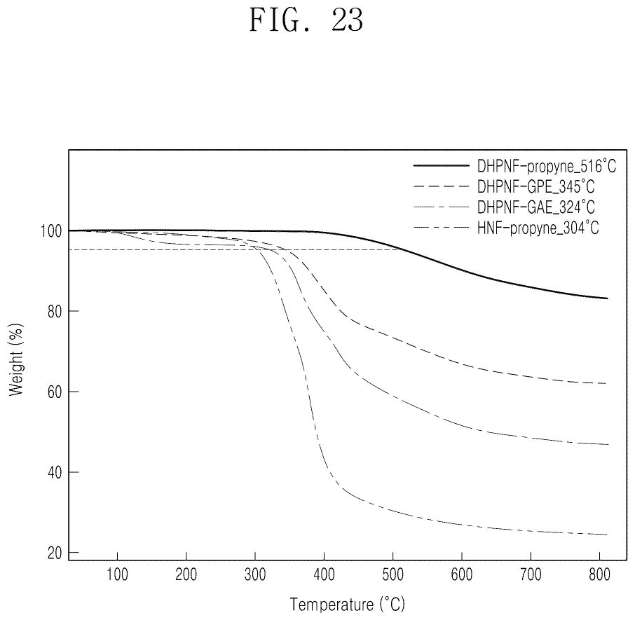

FIG. 23 is a view showing a thermogravimetric analysis graph of HNF-propyne, DHPNF-propyne, DHPNF-GPE, and DHPNF-GAE according to Example 17.

Referring to FIG. 23, it is confirmed that 5 wt % weight loss temperatures (5 wt % T_{d}) are 304° C. for HNF-propyne, 516° C. for DHPNF-propyne, 345° C. for DHPNF-GPE, and 324° C. for DHPNF-GAE. That is, it is confirmed that DHPNF-propyne has the most superior thermal stability.

### [Example 18] Synthesis of Bis-BPF-GPE (Formula 8-1) Introduced with Both a Carbon-Carbon Triple Bond and a Hydroxy Functional Group

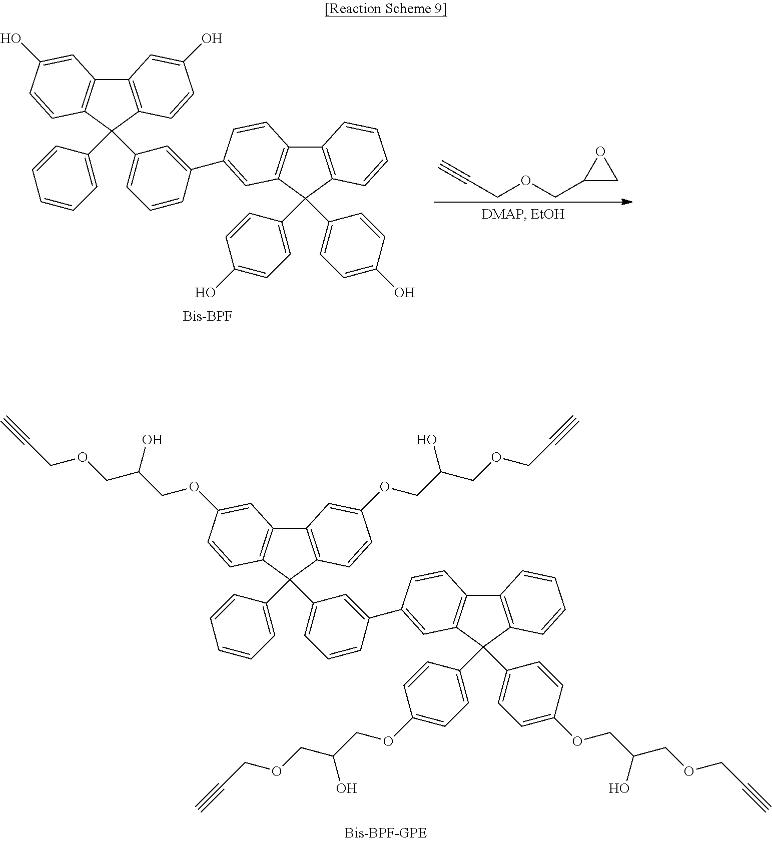

Bis-BPF (0.40 g, 0.57 mmol) and glycidyl propargyl ether (0.52 g, 4.6 mmol) were added to a 25 cm³ seal-tube, and DMAP (14 mg, 0.11 mmol) and ethanol (6 cm³) were further added and sealed to form a reaction solution. The reaction solution was stirred at 90° C. for 12 hours to form a reactant. After the reaction was completed, the reactant was diluted using ethyl acetate (100 cm³). An organic solvent layer of the reactant was washed using water and a saturated aqueous NaCl solution. The organic solvent layer was dried using anhydrous MgSO₄, and the dried organic solvent layer was concentrated under reduced pressure to form a product. The product was diluted with ethyl acetate and then precipitated in hexane. Through filtration, Bis-BPF-GPE (0.50 g, 76%) was obtained as a light yellow solid.

¹H NMR (400 MHz, acetone-d₆): δ=7.94 (d, J=7.9 Hz, 2H, Ar—H), 7.90 (d, J=7.5 Hz, 2H, Ar—H), 7.68 (dd, J=10.2, 2.3 Hz, 4H, Ar—H), 7.45 (dt, J=7.4, 0.9 Hz, 2H, Ar—H), 7.40 (td, J=7.4, 1.2 Hz, 2H, Ar—H), 7.32 (td, J=7.4, 1.2 Hz, 2H, Ar—H), 7.19-7.13 (m, 8H, Ar—H), 6.88-6.82 (m, 8H, Ar—H), 4.21 (d, J=2.4 Hz, 8H, Ar—O—CH₂—), 4.16 (dd, J=5.1, 1.2 Hz, 3H, —OH), 4.11-4.00 (m, 8H, Ar—O—CH₂CHCH₂), 3.95 (dd, J=9.7, 5.9 Hz, 4H, Ar—O—CH₂CH—), 3.65 (qd, J=9.8, 5.3 Hz, 8H, —O—CH₂C≡CH), 2.92 (t, J=2.4 Hz, 3H, —C≡CH)

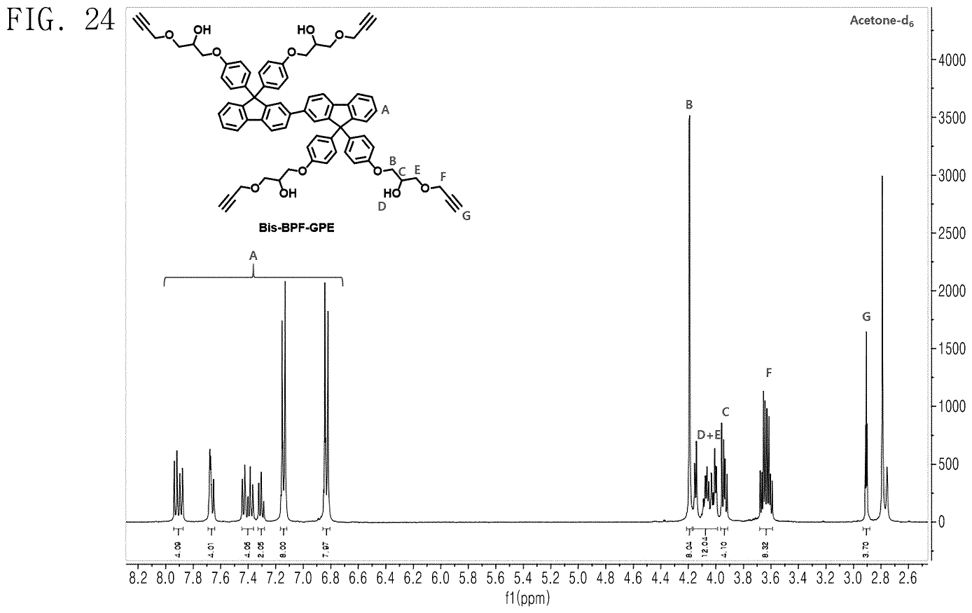

FIG. 24 is a view showing a ¹H NMR graph of Bis-BPF-GPE synthesized according to Example 18.

Referring to FIG. 24, it is confirmed that Bis-BPF-GPE having four carbon-carbon triple bonds and four hydroxyl groups was produced using a ring-opening reaction between Bis-BPF and glycidyl propargyl ether according to Example 18 and Reaction Scheme 9.

### [Example 19] Synthesis of Bis-BPF-GAE (Formula 8-2) Introduced with Both a Carbon-Carbon Double Bond and a Hydroxy Functional Group

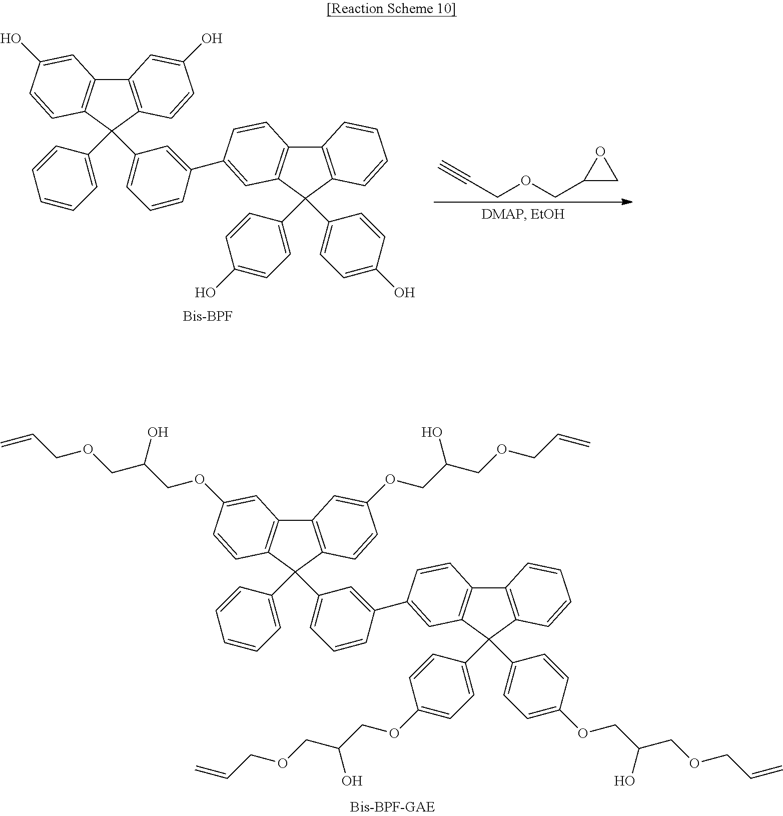

Bis-BPF (0.40 g, 0.57 mmol) and glycidyl allyl ether (0.52 g, 4.6 mmol) were added to a 25 cm³ seal-tube, and DMAP (14 mg, 0.11 mmol) and ethanol (6 cm³) were further added and sealed to form a reaction solution. The reaction solution was stirred at 90° C. for 12 hours to form a reactant. After the reaction was completed, the reactant was diluted using ethyl acetate (100 cm³). An organic solvent layer of the reactant was washed using water and a saturated aqueous NaCl solution. The organic solvent layer was dried using anhydrous MgSO₄, and the dried organic solvent layer was concentrated under reduced pressure to form a product. The product was diluted with ethyl acetate and then added dropwise in hexane to precipitate. Through filtration, Bis-BPF-GAE (0.52 g, 79%) was obtained as a light yellow solid.

¹H NMR (400 MHz, CDCl₃): δ=7.77 (dd, J=8.0, 2.2 Hz, 4H, Ar—H), 7.58-7.54 (m, 4H, Ar—H), 7.40-7.34 (m, 4H, Ar—H), 7.29-7.26 (m, 2H, Ar—H), 7.18-7.12 (m, 8H, Ar—H), 7.18-7.12 (m, 8H, Ar—H), 5.96-5.85 (m, 4H, —CH₂CH═CH₂), 5.28 (ddt, J=17.2, 2.4, 1.5 Hz, 4H, —CH═CH₂), 5.20 (dp, J=10.4, 1.2 Hz, 4H, —CH═CH₂), (4.19-4.12, m, 4H, Ar—OCH₂—CH—), 4.04 (dt, J=6.1, 1.6 Hz, 8H, Ar—O—CH₂—), 4.02-3.98 (m, 8H, —O—CH₂—CH═CH₂), 3.60 (qd, J=9.7, 5.2 Hz, 8H, Ar—CH₂CHCH₂O—)

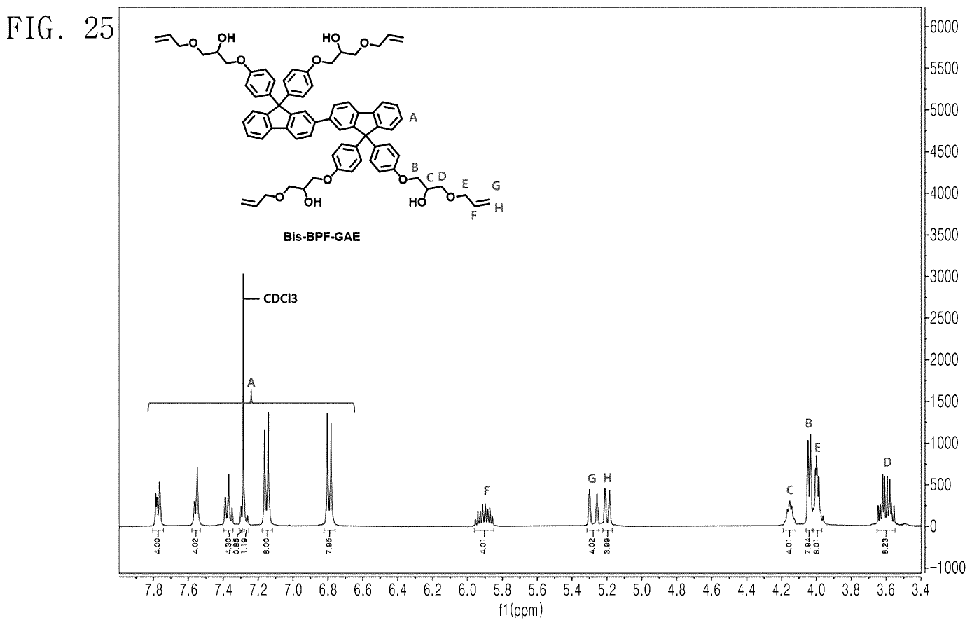

FIG. 25 is a view showing a ¹H NMR graph of Bis-BPF-GAE synthesized according to Example 19.

Referring to FIG. 25, it is confirmed that Bis-BPF-GAE having four carbon-carbon double bonds and four hydroxyl groups was produced using a ring-opening reaction between Bis-BPF and glycidyl allyl ether according to Example 19 and Reaction Scheme 10.

### [Example 20] Synthesis of Bis-BNF-Propyne (Formula 9-1) Introduced with a Carbon-Carbon Triple Bond

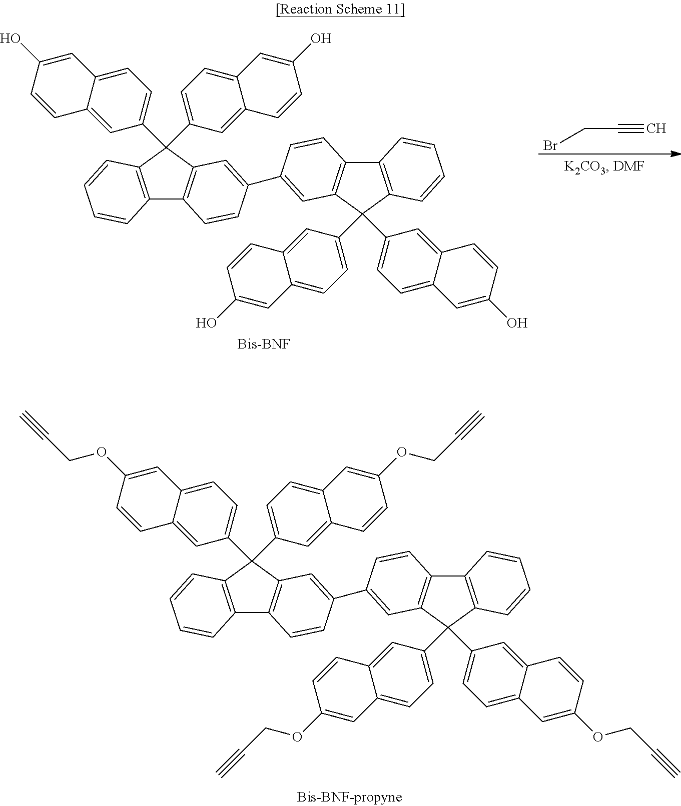

Bis-BNF (0.62 g, 0.97 mmol), propargyl bromide (0.70, 5.9 mmol), and K₂CO₃ (0.81 g, 5.9 mmol) were added to a 250 cm³ round-bottom flask, and DMF (5 cm³) was further added as a solvent to form a reaction solution. The reaction solution was stirred at room temperature for 12 hours to form a reactant. After the reaction was completed, the reactant was diluted using ethyl acetate (100 cm³). An organic solvent layer of the reactant was washed using water and a saturated aqueous NaCl solution. The organic solvent layer was dried using anhydrous MgSO₄, and the dried organic solvent layer was concentrated under reduced pressure to form a product. The product was purified through column chromatography using a mixed solvent of ethyl acetate and hexane (ethyl acetate:hexane=1:3) as a mobile phase, and then concentrated under reduced pressure to obtain Bis-BNF-propyne (0.46 g, 61%) as a light brown solid.

¹H NMR (400 MHz, CDCl₃): δ=7.83 (dd, J=7.7, 2.2 Hz, 4H, Ar—H), 7.69-7.63 (m, 6H, Ar—H), 7.61-7.55 (m, 8H, Ar—H), 7.54-7.49 (m, 4H, Ar—H), 7.44-7.38 (m, 6H, Ar—H), 7.30 (dd, J=7.5, 1.2 Hz, 2H, Ar—H), 7.21 (d, J=2.5 Hz, 4H, Ar—H), 7.13 (dd, J=8.9, 2.5 Hz, 4H, Ar—H), 4.80 (d, J=2.4 Hz, 8H, Ar—O—CH₂—), 2.54 (t, J=2.4 Hz, 4H, —C≡C—H).

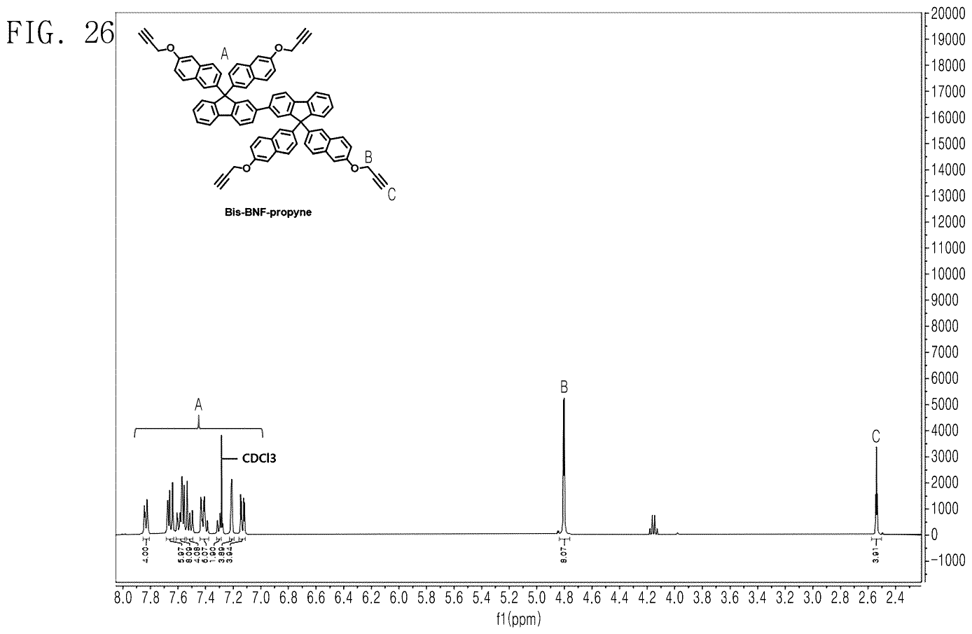

FIG. 26 is a view showing a ¹H NMR graph of Bis-BNF-propyne synthesized according to Example 20.

Referring to FIG. 26, it is confirmed that Bis-BNF-propyne having four carbon-carbon triple bonds was produced through an S_{N}2 reaction between Bis-BNF and propargyl bromide according to Example 20 and Reaction Scheme 11.

### [Example 21] Evaluation of Developer of Bis-BPF-GPE (Formula 8-1) on a Cross-Linked PHS Underlayer

A cross-linked PHS underlayer was formed on a silicon substrate according to Example 14. A Bis-BPF-GPE solution (2.2 wt/vol %) dissolved in MIBK was spin-coated on the underlayer at 2,000 rpm for 60 seconds, and then heated at 80° C. for 1 minute to form a resist thin film (approximately 100 nm thick). The resist thin film was irradiated with UV light in a range of 400 mJ/cm² to 700 mJ/cm² using a 254 nm UV exposure device. Subsequently, a development process using heptanone, MIBK, and nBA as a developer was performed for 30 seconds to form a negative tone pattern.

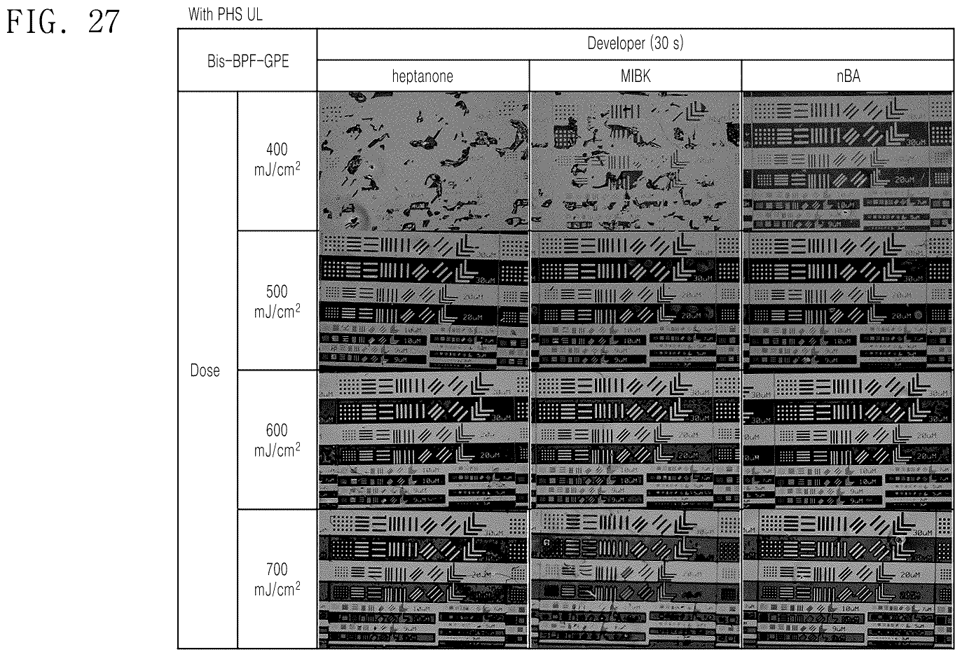

FIG. 27 is an optical microscope image of a negative tone resist pattern formed according to Example 21.

Referring to FIG. 27, a Bis-BPF-GPE resist thin film was formed on a cross-linked PHS underlayer, and a deep UV lithography process was performed on the resist thin film according to Example 21. As a result, it is confirmed that ketone-based and ester-based organic solvents, such as 2-heptanone, n-butyl acetate (nBA), and MIBK, may be used as developers.

### [Example 22] Synthesis of HSnPh₃

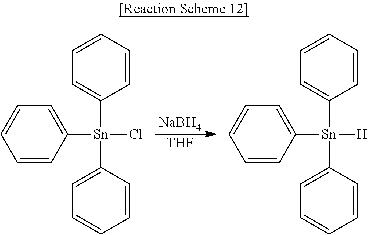

Triphenyltin chloride (2.0 g, 5.19 mmol) and NaBH₄ (0.39 g, 10.4 mmol) were added to a 100 cm³ round-bottom flask (RBF), and THF (20 cm³) was further added as a solvent to form a reaction solution. The reaction solution was stirred at 0° C. for 10 minutes, and then Di water (1 cm²) was added and the mixture was stirred at room temperature for 2 hours. Consequently, a reactant was formed. After the reaction was completed, the reactant was diluted with diethyl ether (EtO₂, 100 cm³). An organic solvent layer of the reactant was washed twice with water, and then washed once more using a saturated aqueous NaCl solution (150 cm³). The organic solvent layer was dried using anhydrous MgSO₄, and the dried organic solvent layer was concentrated under reduced pressure to obtain HSnPh₃ (1.8 g, 97%) as a yellowish transparent liquid.

¹H NMR (400 MHz, CDCl₃, ppm): δ=7.59 (m, 5H, Ar—H), 7.38 (m, 10H, Ar—H), 6.82 (s, Sn—H)

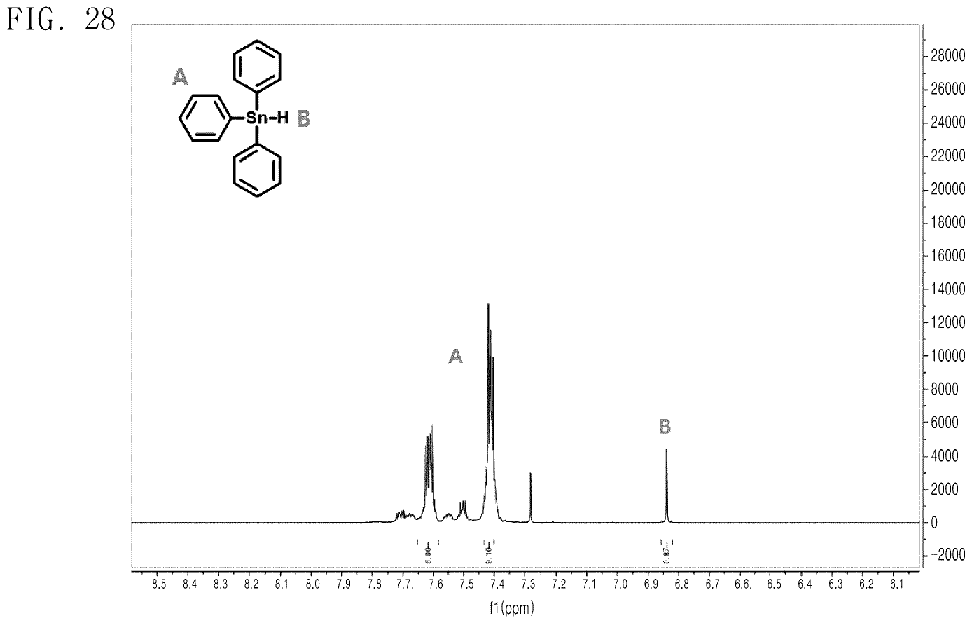

FIG. 28 is a view showing a ¹H NMR graph of HSnPh₃ synthesized according to Example 22.

Referring to FIG. 28, it is confirmed that triphenyltin hydride (HSnPh₃) was formed by reacting the reducing agent NaBH₄ with triphenyltin chloride (ClSnPh₃) according to Example 22 and Reaction Scheme 12.

### [Example 23] Synthesis of HNF-Propyne-HSnPh₃ (Formula 5-2)

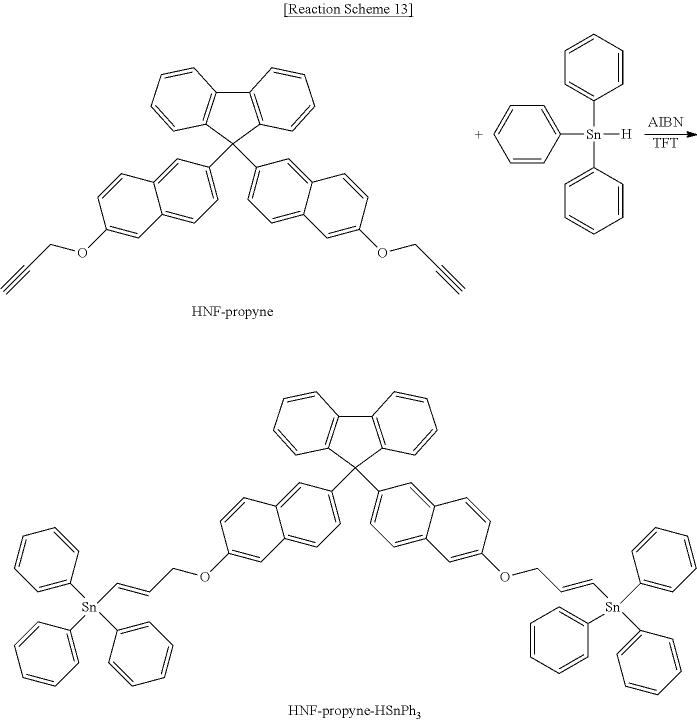

HNF-propyne (0.20 g, 0.38 mmol), HSnPh₃ (0.29 g, 0.83 mmol), and azoisobutyronitrile (AIBN, 6.24 mg, 0.04 mmol) were added to a 100 cm³ 3-neck round-bottom flask, and benzotrifluoride (TFT, 2.5 cm³) was further added as a solvent to form a reaction solution. The reaction solution was stirred under reflux conditions at 110° C. for 12 hours to form a reactant. After the reaction was completed, the reactant was concentrated under reduced pressure, precipitated in methanol, and filtered to form a product. The product was purified through column chromatography using a mixed solvent of chloroform and hexane (chloroform:hexane=3:1) as a mobile phase, and then concentrated under reduced pressure to obtain HNF-propyne-HSnPh₃ (0.33 g, 71%) as a solid.

¹H NMR (400 MHz, acetone-d6): δ=7.64-7.58 (m, 10H, Ar—H), 7.57-7.52 (m, 10H, Ar—H), 7.40-7.31 (m, 10H, Ar—H), 4.89-4.68 (m, 4H, Ar—H), 4.89-4.87 (m, 2H, —CH═CH—), 4.72-4.66 (d, J=4.8 Hz, 2H, —CH═CH—), 2.79 (s, 4H, —O—CH₂—)/m/z (ESI-TOF-MS) 1231.72485 ([M+H]⁺. C₇₅H₅₈O₂Sn₂H requires M, 1229.69)/Anal. Found: C, 73.5; H, 4.7. Calc. for C₇₅H₅₈O₂Sn₂: C, 73.3; H, 4.8%.

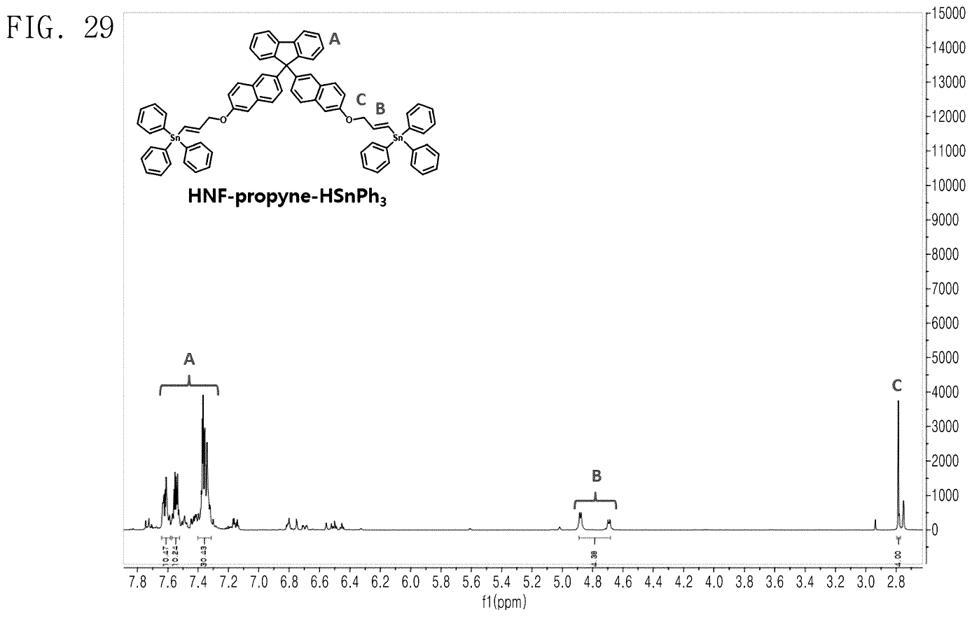

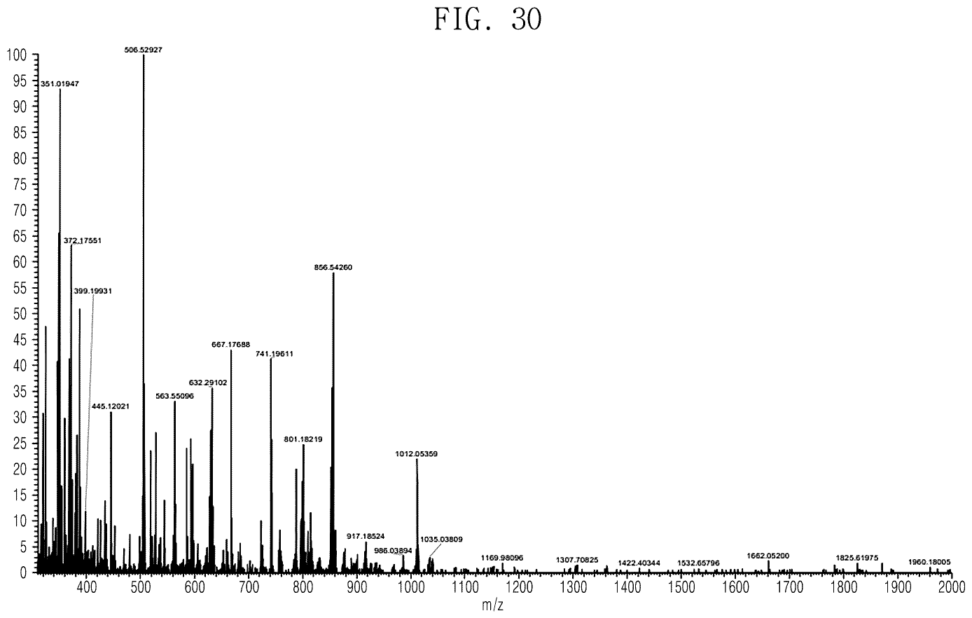

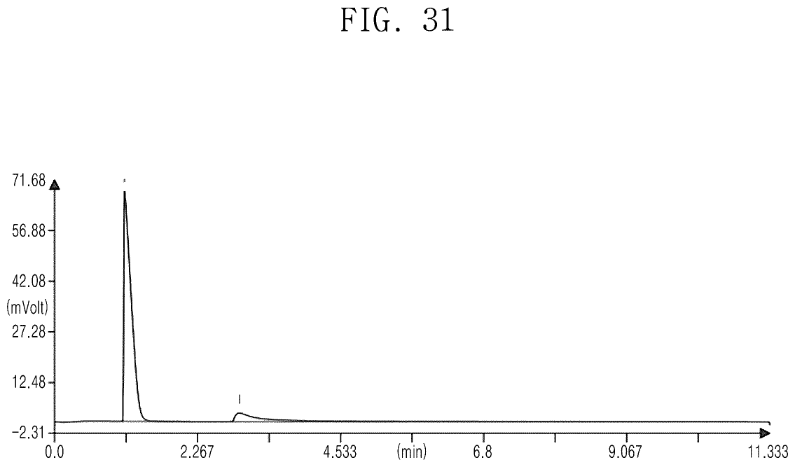

FIG. 29 is a view showing a ¹H NMR graph of HNF-propyne-HSnPh₃ synthesized according to Example 23. FIG. 30 is a view showing a mass spectrometry graph of HNF-propyne-HSnPh₃ synthesized according to Example 23, and FIG. 31 is a view showing an elemental analysis graph of HNF-propyne-HSnPh₃ synthesized according to Example 23.

Referring to FIGS. 29 to 31, it is confirmed that HNF-propyne-HSnPh₃ introduced with a triphenyltin unit is formed through a hydrostannylation reaction in which HSnPh₃ is subjected to a reaction with the carbon-carbon triple bond of HNF-propyne using azoisobutyronitrile (AIBN) as an initiator according to Example 23 and Reaction Scheme 13.

### [Example 24] Deep UV Lithography Evaluation of HNF-Propyne-HSnPh₃ (Formula 5-2)

A solution of HNF-propyne-HSnPh₃ (2.5 wt %) dissolved in n-butyl acetate (nBA) was spin-coated on a silicon substrate at 2,000 rpm for 60 seconds, and then heated at 80° C. for 1 minute to form a resist thin film (approximately 90 nm thick). The resist thin film was irradiated with 254 nm UV light at a dose of 1.4 J/cm², and subjected to a development process performed using a mixed solvent of methyl isobutyl ketone (MIBK) and methyl isobutyl carbinol (MIBC) (MIBK:MIBC=1:4) as a developer for 10 seconds to form a negative tone resist pattern.

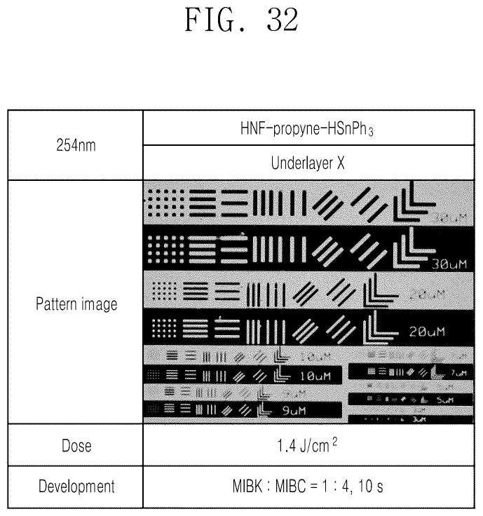

FIG. 32 is an optical microscope image of a negative tone resist pattern formed by a deep UV lithography process according to Example 24.

Referring to FIG. 32, it is confirmed that a negative tone resist pattern is formed by performing deep UV lithography and development processes on a resist thin film formed using an HNF-propyne-HSnPh₃ solution according to Example 24. Accordingly, it is confirmed that a resist compound containing an organic monomer having a carbon-carbon double bond and a triphenyltin unit may form cross-linking bonds between organic molecules through a radical reaction under 254 nm UV irradiation.

### [Example 25] Deep UV Lithography Evaluation of HNF-Propyne-HSnPh₃ (Formula 5-2) on a Cross-Linked PHS Underlayer

A cross-linked PHS underlayer was formed on a silicon substrate according to Example 14. A solution of HNF-propyne-HSnPh₃ (2.5 wt %) dissolved in n-butyl acetate (nBA) was spin-coated on the underlayer at 2,000 rpm for 60 seconds, and then heated at 80° C. for 1 minute to form a resist thin film (approximately 90 nm thick). The resist thin film was irradiated with 254 nm UV light at a dose of 1.4 J/cm², and subjected to a development process performed using a mixed solvent of methyl isobutyl ketone (MIBK) and methyl isobutyl carbinol (MIBC) (MIBK:MIBC=1:4) as a developer for 10 seconds to form a negative tone resist pattern. In addition, the resist thin film was irradiated with 254 nm UV light at a dose of 1.4 J/cm², and subjected to a development process performed using a mixed solvent of MIBK and MIBC (MIBK:MIBC=1:5) as a developer for 30 seconds to form a negative tone resist pattern.

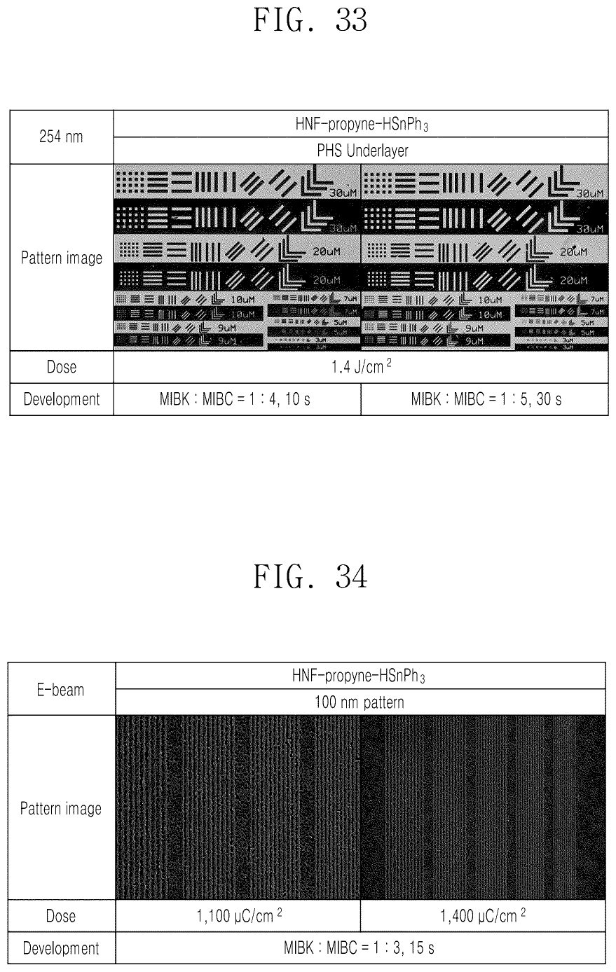

FIG. 33 is an optical microscope image of a negative tone resist pattern formed by a deep UV lithography process according to Example 25.

Referring to FIG. 33, it is confirmed that a negative tone resist pattern is formed by forming an HNF-propyne-HSnPh₃ resist thin film on the cross-linked PHS underlayer and performing deep UV lithography and development processes on the resist thin film according to Example 25.

### [Example 26] E-Beam Lithography Evaluation of HNF-Propyne-HSnPh₃ (Formula 5-2)

A solution of HNF-propyne-HSnPh₃ (2.5 wt %) dissolved in nBA was spin-coated on a silicon substrate at 2,000 rpm for 60 seconds, and then heated at 80° C. for 1 minute to form a resist thin film (approximately 90 nm thick). The resist thin film was irradiated with an e-beam of 1,100 μC/cm² to 1,400 μC/cm², and subjected to a development process performed using a mixed solvent of MIBK and IPA (MIBK:IPA=1:3) as a developer for 15 seconds to form a negative tone resist pattern.

FIG. 34 is a scanning electron microscope image of a negative tone resist pattern formed by an e-beam lithography process according to Example 26.

Referring to FIG. 34, it is confirmed that a negative tone resist pattern is formed by performing e-beam lithography and development processes on a resist thin film formed using an HNF-propyne-HSnPh₃ solution according to Example 26. Accordingly, it is confirmed that a resist compound containing an organic monomer having a carbon-carbon double bond and a triphenyltin unit may form cross-linking bonds between organic molecules through a radical reaction under e-beam irradiation.

### [Example 27] Synthesis of DMPNF-Propyne (Formula 7-4)

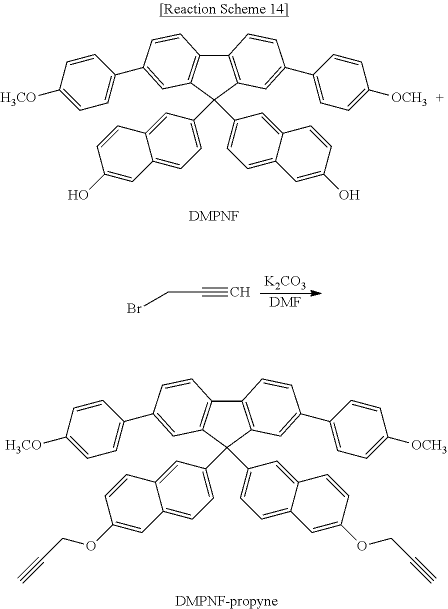

DMPNF (3 g, 4.53 mmol), propargyl bromide (1.55 g, 10.41 mmol), and K₂CO₃ (1.88 g, 13.58 mmol) were added to a 100 cm³ round-bottom flask, and dimethylformamide (DMF, 30 cm³) was further added as a solvent to form a reaction solution. The reaction solution was stirred at room temperature for 12 hours to form a reactant. After the reaction was completed, the reactant was diluted with dichloromethane (150 cm³). An organic solvent layer of the reactant was washed twice with water and further washed with a saturated aqueous NaCl solution (150 cm³). The organic solvent layer was dried using anhydrous MgSO₄, and the dried organic solvent layer was concentrated under reduced pressure to form a product. The product was purified through column chromatography using chloroform as a mobile phase, and then concentrated under reduced pressure to obtain DMPNF-propyne (2.2 g, 66%) as a white solid.

¹H NMR (400 MHz, CDCl₃): δ=8.01 (s, 1H, Ar—H), 7.99 (s, 1H, Ar—H), 7.85 (s, 2H, Ar—H), 7.78 (s, 1H, Ar—H), 7.76 (s, 3H, Ar—H), 7.70 (d, 1H, J=1.6 Hz, Ar—H), 7.69 (s, 1H, Ar—H), 7.68 (d, 1H, J=1.6 Hz, Ar—H), 7.67 (s, 1H, Ar—H), 7.56 (d, 6H, J=8.8 Hz, Ar—H), 7.37 (d, 2H, J=2.4 Hz, Ar—H), 7.12 (dd, 2H, J=2.4, 2.4 Hz, Ar—H), 6.96 (d, 4H, J=8.8 Hz, Ar—H), 4.88 (d, 4H, J=2.4 Hz, —OCH₂—), 3.80 (s, 6H, —OCH₃), 3.06 (s, 3H, —C≡CH)/m/z (ESI-TOF-MS) 738.2767 ([M+H]⁺. C₅₃H₃₈O₄H requires M, 739.88).

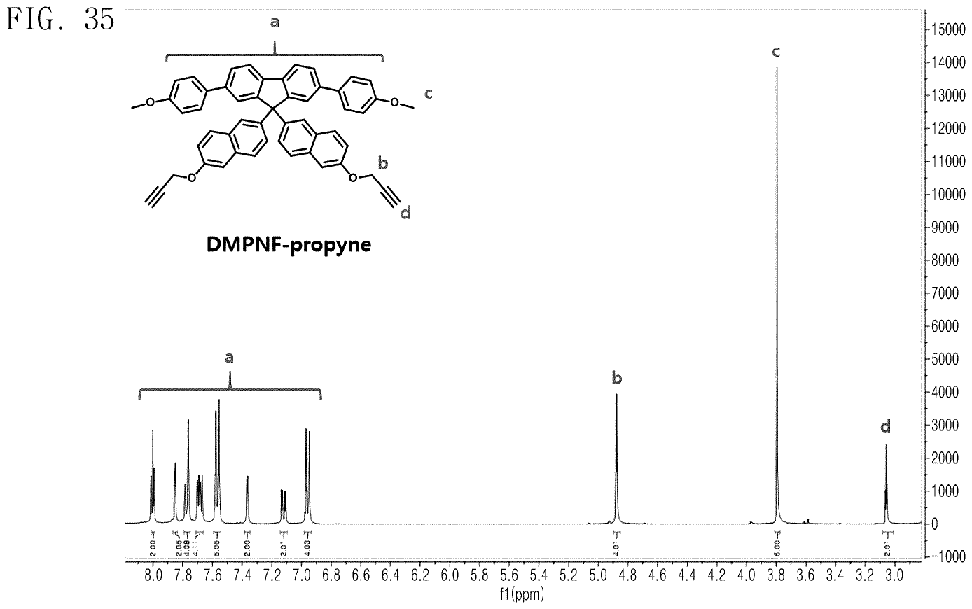

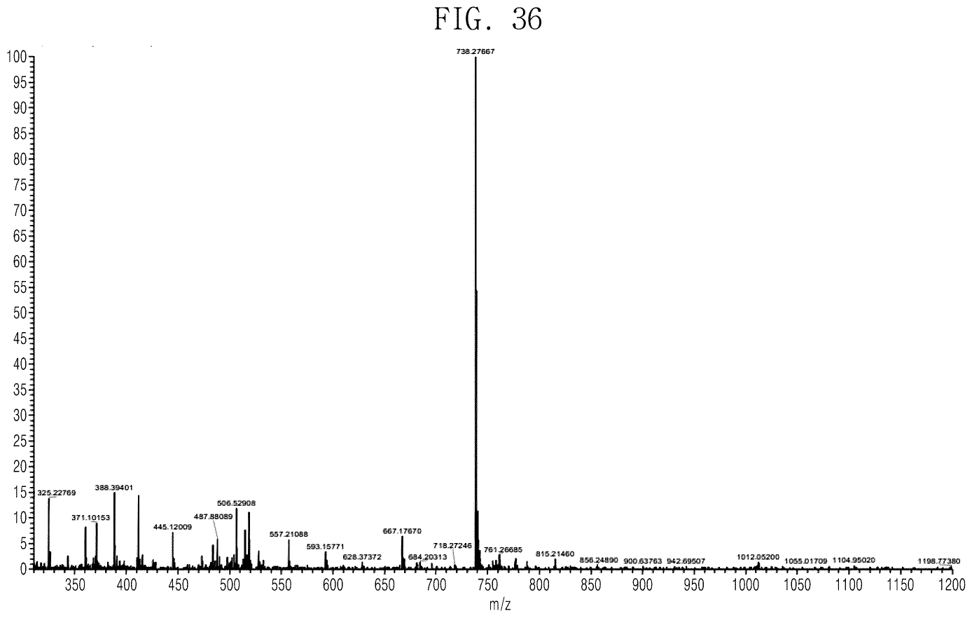

FIG. 35 is a view showing a ¹H NMR graph of DMPNF-propyne synthesized according to Example 27, and FIG. 36 is a view showing a mass spectrometry graph of DMPNF-propyne synthesized according to Example 27.

Referring to FIGS. 35 and 36, it is confirmed that DMPNF-propyne containing a carbon-carbon triple bond is formed by reacting an —OH unit of DMPNF with propargyl bromide using K₂CO₃ as a base, according to Example 27 and Reaction Scheme 14.

### [Example 28] Synthesis of DMPNF-Propyne-HSnPh₃ (Formula 7-5)

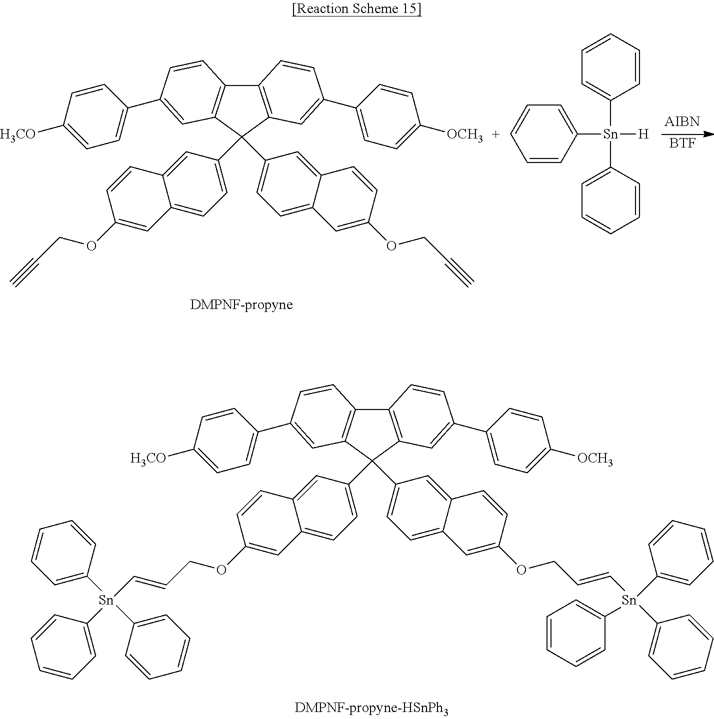

DMPNF-propyne (1.00 g, 1.35 mmol), triphenyltin hydride (1.05 g, 2.98 mmol), and AIBN (11.1 mg, 0.05 mmol) were added to a 100 cm³ 3-neck round-bottom flask, and benzotrifluoride (BTF, 12 cm³) was further added as a solvent to form a reaction solution. The reaction solution was stirred under reflux conditions at 110° C. for 12 hours to form a reactant. After the reaction was completed, the reactant was concentrated under reduced pressure and diluted with ethyl acetate. Subsequently, the reactant was precipitated in methanol and filtered to form a product. The product was purified through column chromatography using a mixed solvent of dichloromethane and hexane (dichloromethane:hexane=4:1) as a mobile phase, and concentrated again under reduced pressure to obtain DMPNF-propyne-HSnPh₃ (0.4 g, 21%) as a light green solid.

¹H NMR (400 MHz, acetone-d6): δ=8.03-7.97 (m, 2H, Ar—H), 7.88-7.78 (m, 2H, Ar—H), 7.70-7.64 (m, 4H, Ar—H), 7.64-7.50 (m, 20H, Ar—H), 7.49-7.41 (m, 4H, Ar—H), 7.37-7.31 (m, 18H, Ar—H), 7.21-7.11 (m, 2H, Ar—H), 7.00-6.93 (m, 4H, Ar—H), 6.84-6.40 (m, 4H, —OCH₂—), 4.90-4.84 (m, 2H, —CH═CH—), 4.71-4.65 (m, 2H, —CH═CH—), 3.80 (s, 6H, —OCH₃)/m/z (ESI-TOF-MS) 1445.64087 ([M+H]⁺. C₈₉H₇₀O₄Sn₂H requires M, 1441.94)./Anal. Found: C, 73.2; H, 4.8. Calc. for C₈₉H₇₀O₄Sn₂: C, 74.2; H, 4.9%.

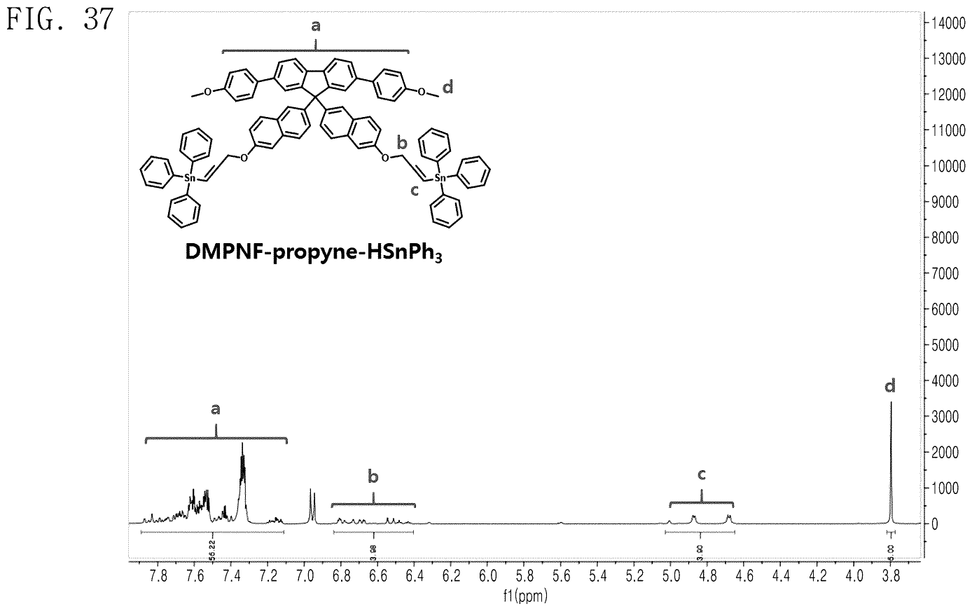

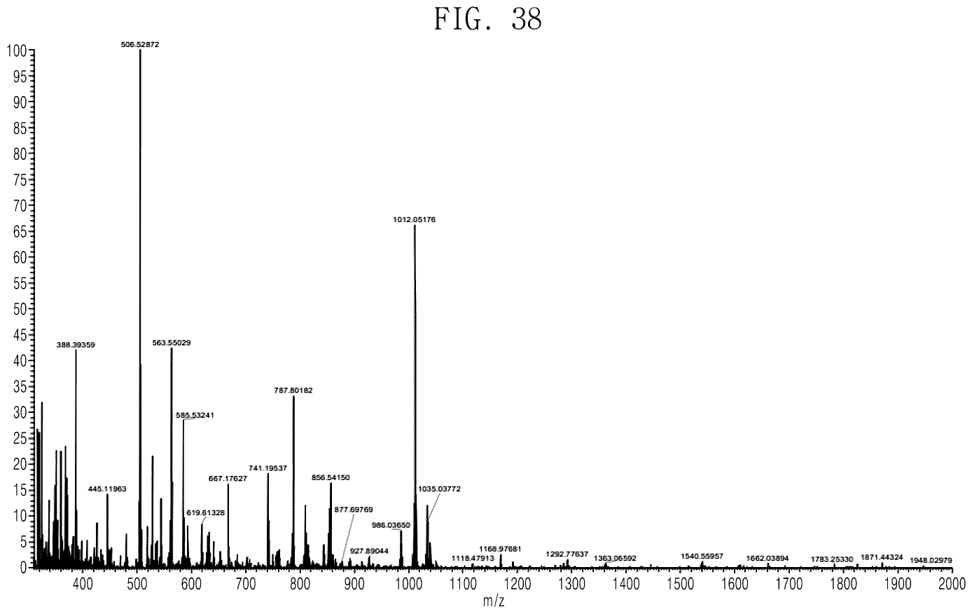

FIG. 37 is a view showing a ¹H NMR graph of DMPNF-propyne-HSnPh₃ synthesized according to Example 28. FIG. 38 is a view showing a mass spectrometry graph of DMPNF-propyne-HSnPh₃ synthesized according to Example 28, and FIG. 39 is a view showing an elemental analysis graph of DMPNF-propyne-HSnPh₃ synthesized according to Example 28.

Referring to FIGS. 37 to 39, it is confirmed that DMPNF-propyne-HSnPh₃ introduced with a triphenyltin unit is formed through a hydrostannylation reaction in which HSnPh₃ is subjected to a reaction with the carbon-carbon triple bond of DMPNF-propyne using azoisobutyronitrile (AIBN) as an initiator according to Example 28 and Reaction Scheme 15.

### [Example 29] Deep UV Lithography Evaluation of DMPNF-Propyne-HSnPh₃ (Formula 7-5)

A cross-linked PHS underlayer was formed on a silicon substrate according to Example 14.

A solution of DMPNF-propyne-HSnPh₃ (2.5 wt %) dissolved in nBA was spin-coated on the underlayer at 2,500 rpm for 60 seconds, and then heated at 80° C. for 1 minute to form a resist thin film (approximately 90 nm thick). The resist thin film was irradiated with 254 nm UV light at a dose of 700 mJ/cm², and subjected to a development process performed using cyclohexane as a developer for 15 seconds to form a negative tone resist pattern. In addition, the resist thin film was irradiated with 254 nm UV light at a dose of 1.4 J/cm², and subjected to a development process performed using a mixed solvent of MIBK and MIBC (MIBK:MIBC=1:4) as a developer for 60 seconds to form a negative tone resist pattern.

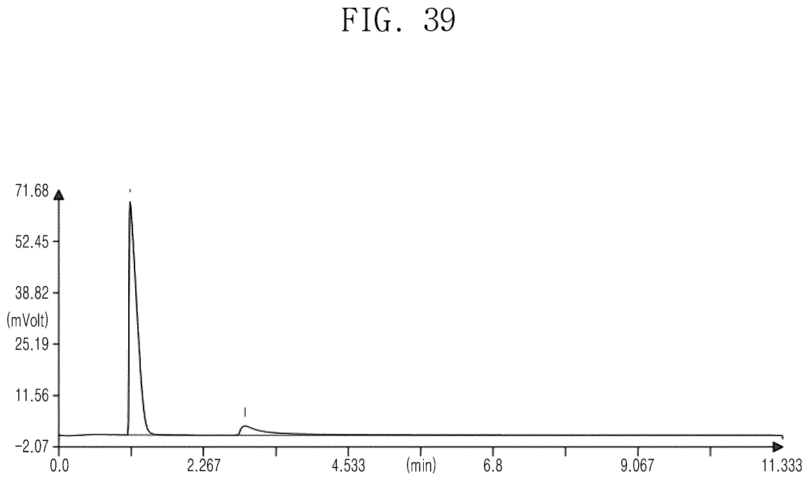

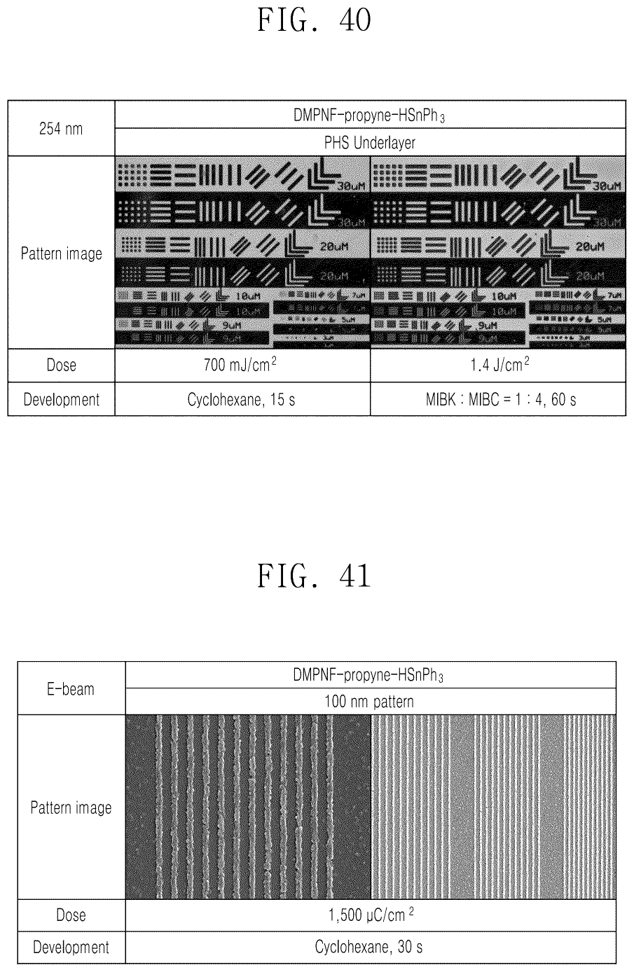

FIG. 40 is an optical microscope image of a negative tone resist pattern formed by a deep UV lithography process according to Example 29.

Referring to FIG. 40, it is confirmed that a negative tone resist pattern is formed by forming a DMPNF-propyne-HSnPh₃ resist thin film on a cross-linked PHS underlayer and performing deep UV lithography and development processes on the resist thin film according to Example 29.

### [Example 30] E-Beam Lithography Evaluation of DMPNF-Propyne-HSnPh₃ (Formula 7-5)

A cross-linked PHS underlayer was formed on a silicon substrate according to Example 14.

A solution of DMPNF-propyne-HSnPh₃ (2.5 wt %) dissolved in nBA was spin-coated on the underlayer at 2,500 rpm for 60 seconds, and then heated at 80° C. for 1 minute to form a resist thin film (approximately 90 nm thick). The resist thin film was irradiated with an e-beam of 1,500 μC/cm², and subjected to a development process performed using cyclohexane as a developer for 30 seconds to form a negative tone resist pattern.

FIG. 41 is a scanning electron microscope image of a negative tone resist pattern formed by an e-beam lithography process according to Example 30.

Referring to FIG. 41, it is confirmed that a negative tone resist pattern is formed by forming a DMPNF-propyne-HSnPh₃ resist thin film on a cross-linked PHS underlayer and performing e-beam lithography and development processes on the resist thin film according to Example 30.

### [Example 31] Evaluation of Change in Solubility of DMPNF-Propyne-HSnPh₃ (Formula 7-5) with Respect to e-Beam

A cross-linked PHS underlayer was formed on a silicon substrate according to Example 14.

A solution of DMPNF-propyne-HSnPh₃ (2.5 wt %) dissolved in nBA was spin-coated on the underlayer at 2,500 rpm for 60 seconds, and then heated at 80° C. for 1 minute to form a resist thin film (approximately 90 nm thick). The resist thin film was irradiated with an e-beam having an energy range of 100 μC/cm² to 3,000 μC/cm², and subjected to a development process performed using cyclohexane as a developer for 10 seconds to form a negative tone resist pattern. A thickness of the resist pattern was measured using an Alpha-Step® D-300 stylus profiler manufactured by KLA-Tencor. Consequently, a solubility change curve (contrast curve) according to e-beam irradiation dose was obtained.

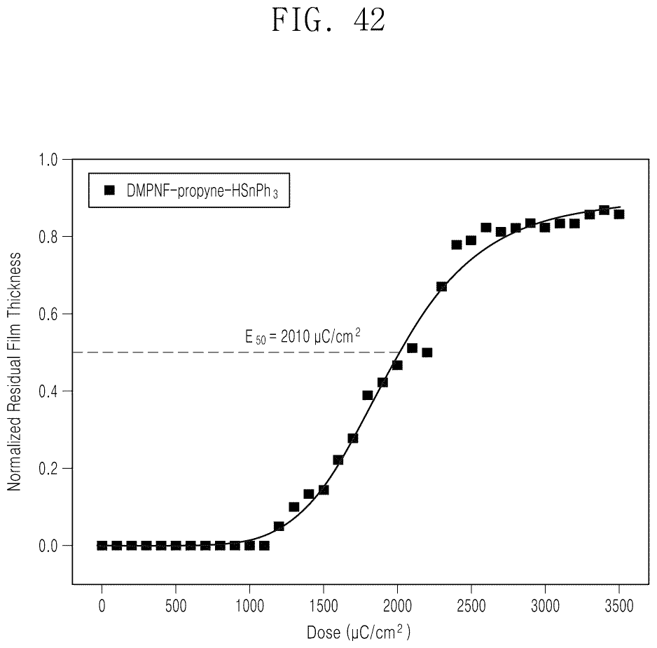

FIG. 42 is a graph showing the results of evaluating solubility of a resist thin film for an e-beam lithography process according to Example 31.

Referring to FIG. 42, it is confirmed that when a DMPNF-propyne-HSnPh₃ resist thin film is irradiated with an e-beam of 2,010 μC/cm², the thickness of the resist pattern is maintained at approximately 50% of the thickness of the initially coated resist thin film.

### [Example 32] Evaluation of Change in Solubility of DMPNF-Propyne-HSnPh₃ (Formula 7-5) with Respect to Extreme UV

A cross-linked PHS underlayer was formed on a silicon substrate according to Example 14.

A solution of DMPNF-propyne-HSnPh₃ (2.5 wt %) dissolved in nBA was spin-coated on the underlayer at 2,500 rpm for 60 seconds, and then heated at 80° C. for 1 minute to form a resist thin film (approximately 90 nm thick). The resist thin film was irradiated with EUV having an energy range of 5 mJ/cm² to 125 mJ/cm², and subjected to a development process performed using cyclohexane as a developer for 10 seconds to form a negative tone resist pattern. A thickness of the resist pattern was measured using an Alpha-Step® D-300 stylus profiler manufactured by KLA-Tencor. Consequently, a solubility change curve (contrast curve) according to EUV irradiation dose was obtained.

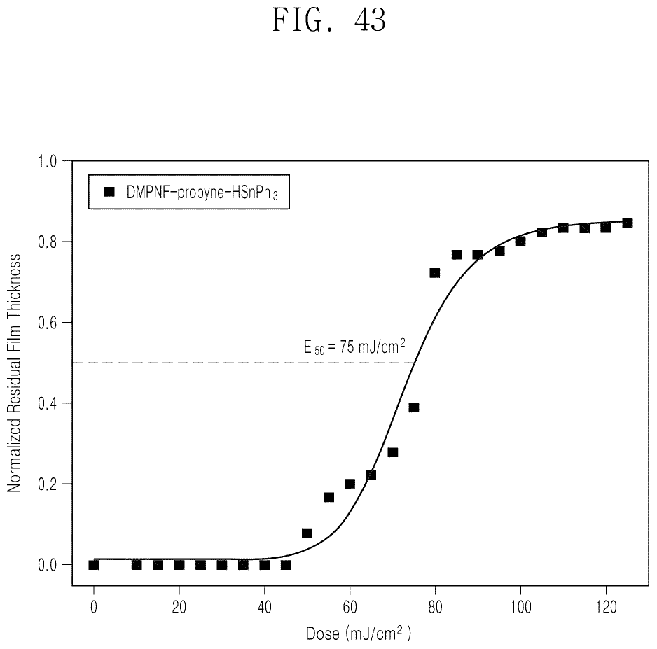

FIG. 43 is a graph showing the results of evaluating solubility of a resist thin film for an extreme UV lithography process according to Example 32.

Referring to FIG. 43, it is confirmed that when a DMPNF-propyne-HSnPh₃ resist thin film was irradiated with an extreme UV of 75 mJ/cm², the thickness of the resist pattern is maintained at approximately 50% of the thickness of the initially coated resist thin film.

### A Method for Manufacturing a Semiconductor Device Using a Resist Compound According to Embodiments of the Inventive Concept Will be Described.

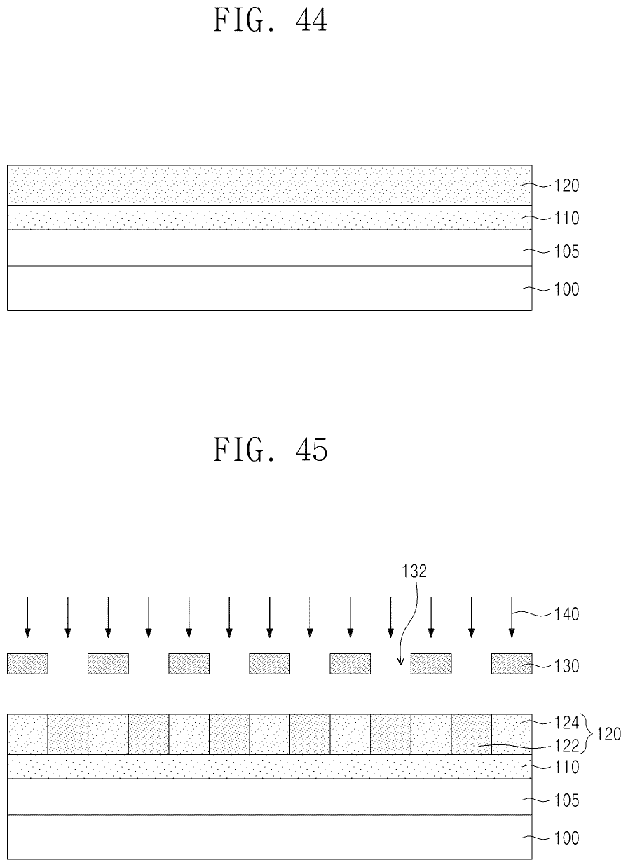

FIGS. 44 to 47 are cross-sectional views showing a method for manufacturing a semiconductor device using a resist composition according to embodiments of the inventive concept.

Referring to FIG. 44, an etching target film 105 may be formed on a substrate 100, and a photoresist film 120 may be formed on the etching target film 105. According to some embodiments, an underlayer 110 may be formed between the etching target film 105 and the photoresist film 120, but an embodiment of the inventive concept is not limited thereto.

The substrate 100 may be a semiconductor substrate, and for example, may be a silicon substrate, a germanium substrate, or a silicon/germanium substrate. The etching target film 105 may be formed of any one selected from or a semiconductor material, a conductive material, and an insulating material, a combination thereof. The etching target film 105 may be formed as a single film, or may include a plurality of films stacked on the substrate 100. The underlayer 110 may include, for example, 1,3-divinyl-1,1,3,3-tetramethyldisilazane or cross-linked poly(4-hydroxystyrene) (PHS).

The photoresist film 120 may include the resist compound according to embodiments of the inventive concept. The resist compound may include the organic molecule, and the organic molecule may include the core structure containing at least six benzene rings and the functional group bonded to at least one of the benzene rings. The functional group may be represented by Formula 1 or Formula 2. The forming of the photoresist film 120 may include, for example, applying the resist compound onto the etching target film 105 (or on the underlayer 110). The applying of the resist compound is performed, for example, through a spin coating method. The forming of the photoresist film 120 may further include, for example, performing a heat treatment process (e.g., a soft baking process) on the applied resist compound.

Referring to FIG. 45, an exposure process may be performed on the photoresist film 120. The exposure process may include irradiating the photoresist film 120 with light 140. The light 140 may be deep UV, extreme UV, or e-beams. For example, the exposure process may include aligning a photomask 130 on the photoresist film 120, and irradiating the photoresist film 120 with the light 140 (e.g., deep UV or extreme UV) through the photomask 130. As another example, the exposure process may include irradiating and scanning the photoresist film 120 with the light 140 (e.g., e-beams) using an e-beam lithography device.

The photoresist film 120 may include a first portion 122 exposed to the light 140 and a second portion 124 not exposed to the light 140. For example, the light 140 may be applied to the first portion 122 through an opening 132 of the photomask 130, and may be blocked by the photomask 130 and thus not applied to the second portion 124.

When the light 140 is applied to the resist compound, a C═C bond or a C≡C bond of an R1 group of the functional group represented by Formula 1 or Formula 2 may readily accept secondary electrons generated by photons, and accordingly, radicals (e.g., carbon radicals) may be generated. The organic molecules adjacent to each other may be cross-linked through a bond between the radicals, or a bond between the C═C bond or C≡C bond of the R1 group of the functional group and the radicals, and consequently, the resist compound may exhibit reduced solubility by irradiation with the light 140.

In the first portion 122 of the photoresist film 120, the resist compound may include the radicals (e.g., carbon radicals) generated by irradiation with the light 140, and may include a structure in which the organic molecules are bonded (or cross-linked) to each other through the radicals. In the second portion 124 of the photoresist film 120, the chemical structure of the resist compound may not change, and the resist compound may include an organic monomer structure. Accordingly, the first portion 122 and the second portion 124 of the photoresist film 120 may have different chemical structures due to the exposure process, which may consequently result in a difference in solubility between the first portion 122 and the second portion 124.

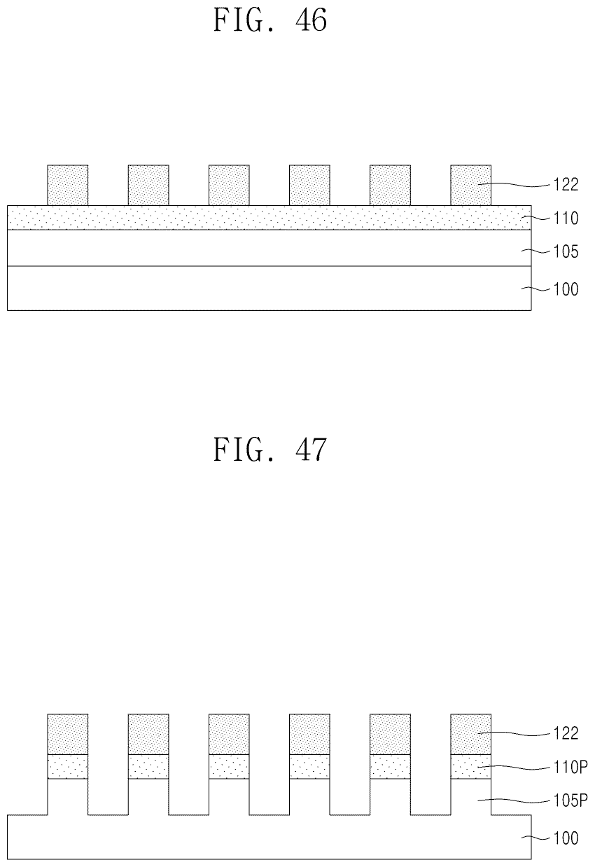

Referring to FIG. 46, after the exposure process, the photomask 130 may be removed. A development process may be performed on the exposed photoresist film 120. The performing of the development process may include removing the second portion 124 of the photoresist film 120 using a developer. The developer may include, for example, a benzene-based organic solvent such as toluene and o-xylene. As another example, the developer may include ketone-based and ester-based organic solvents such as 2-heptanone, n-butyl acetate (nBA), and MIBK.

Through the development process, the second portion 124 of the photoresist film 120 may be selectively removed. The first portion 122 of the photoresist film 120 may be referred to as a photoresist pattern, and the photoresist pattern 122 may be a negative tone pattern.

Referring to FIG. 47, the underlayer 110 and the etching target film 105 may be etched using the photoresist pattern 122 as an etching mask. The etching of the underlayer 110 and the etching target film 105 may include, for example, performing a wet or dry etching process. As the etching target film 105 is etched, a target pattern 105P may be formed. According to some embodiments, the residue of the underlayer 110 may remain between the photoresist pattern 122 and the target pattern 105P. After the target pattern 105P is formed, the photoresist pattern 122 may be removed. The target patterns 105P may be semiconductor patterns, conductive patterns, or insulation patterns in a semiconductor device.

According to an aspect of the inventive concept, a resist compound may include an organic molecule having a smaller molecular weight than that of a polymer, and thus photoresist patterns formed using the resist compound may exhibit reduced line width roughness and increased resolution. In addition, the organic molecule may include a core structure containing at least six benzene rings, and thus the photoresist film formed using the resist compound may exhibit increased etch resistance. Furthermore, the organic molecule may include a functional group represented by Formula 1 or Formula 2, a C═C bond or a C≡C bond of an R1 group of the functional group may readily accept secondary electrons generated by photons, and accordingly, the photoresist film may exhibit increased sensitivity.

The R1 group of the functional group may have a substituent represented by Formula 3. In this case, the etch resistance of the photoresist film may be further increased by a metal element of the substituent. Moreover, the metal element (e.g., tin) may have high absorption characteristics for extreme UV, which may consequently lead to a further increase in the sensitivity of the photoresist film.

When the etch resistance of the photoresist film is increased, the photoresist patterns may be formed to have a relatively small aspect ratio. Accordingly, pattern collapse of the photoresist patterns may be minimized during a development process.

Consequently, a resist compound capable of improving the resolution and sensitivity of photoresist patterns, increasing the etch resistance of the photoresist patterns, and inhibiting the collapse of the photoresist patterns, and a method for manufacturing a semiconductor device using the same may be provided.

The above description on embodiments of the present disclosure provides examples for describing the present disclosure. Thus, the present disclosure is not limited to the above-described embodiments, and it would be clarified that various modifications and changes, for example, combinations of the above embodiments, could be made by those skilled in the art within the technical spirit of the present disclosure.

What is claimed is:

1. A resist compound for photolithography, comprising an organic molecule,
wherein the organic molecule include:
a core structure containing at least six benzene rings; and
a functional group bonded to at least one of the benzene rings,
wherein the functional group is represented by Formula 1 or Formula 2: 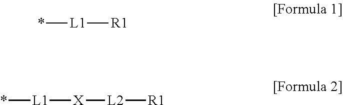
wherein in Formulas 1 and 2 above,
* is a portion bonded to a carbon of the at least one of the benzene rings,
L1 and L2 are each independently an alkylene group having 1 to 10 carbon atoms, or oxygen,
X is an alkylene group having 1 to 10 carbon atoms in which at least one hydrogen is substituted with a hydroxyl group (—OH),
R1 is an unsaturated hydrocarbon group having 2 to 10 carbon atoms with at least one C═C bond or at least one C≡C bond, or an unsaturated hydrocarbon group having 2 to 10 carbon atoms with at least one C═C bond and with at least one hydrogen substituted with a substituent represented by Formula 3: 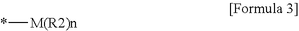
wherein in Formula 3 above, * is a portion bonded to a carbon of R1, M is a metal, R2 is an aryl group having 6 to 30 carbon atoms, and n is an integer of 1 to 3.

2. The resist compound for photolithography of claim 1, wherein in Formulas 1 and 2 above, L1 and L2 are each independently oxygen.

3. The resist compound for photolithography of claim 2, wherein in Formulas 1 and 2 above, R1 is represented by Formula 4-1, Formula 4-2, or Formula 4-3: 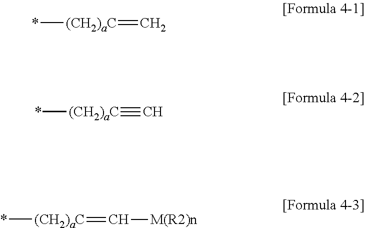
wherein in Formulas 4-1, 4-2, and Formula 4-3 above, * is a portion bonded to oxygen of Formula 1 or Formula 2, and a is an integer of 1 to 8, and
in Formula 4-3 above, M, R2, and n are as defined in Formula 3 above.

4. The resist compound for photolithography of claim 1, wherein in Formulas 3 above, M is one selected from the group consisting of tin (Sn), iodine (I), zinc (Zn), hafnium (Hf), zirconium (Zr), lithium (Li), sodium (Na), potassium (K), beryllium (Be), magnesium (Mg), calcium (Ca), barium (Ba), aluminum (Al), silicon (Si), cadmium (Cd), mercury (Hg), chromium (Cr), iron (Fe), cobalt (Co), nickel (Ni), copper (Cu), germanium (Ge), palladium (Pd), platinum (Pt), lead (Pb), strontium (Sr), and manganese (Mn).

5. The resist compound for photolithography of claim 1, wherein the substituent represented by Formula 3 above comprises a substituent represented by Formula 3-1: 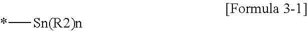
wherein in Formula 3-1 above, * is a portion bonded to a carbon of R1, R2 is a phenyl group, and n is an integer of 1 to 3.

6. The resist compound for photolithography of claim 1, wherein the organic molecule is represented by Formula 5: 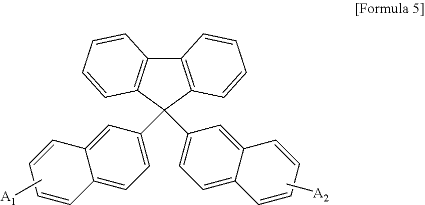
wherein in Formula 5 above, A1 and A2 are each independently the functional group represented by Formula 1 or Formula 2 above.

7. The resist compound for photolithography of claim 6, wherein in Formulas 1 and 2 above, L1 and L2 are each independently oxygen, and
in Formula 3 above, M is tin.

8. The resist compound for photolithography of claim 1, wherein the organic molecule is represented by Formula 7: 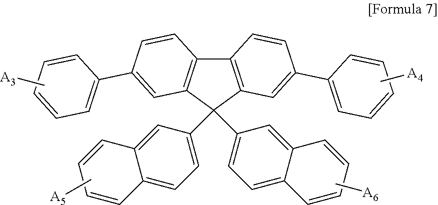
wherein in Formula 7 above,
A3 and A4 are each independently an alkoxy group having 1 to 10 carbon atoms, or the functional group represented by Formula 1 or Formula 2 above, and
A5 and A6 are each independently the functional group represented by Formula 1 or Formula 2 above.

9. The resist compound for photolithography of claim 8, wherein in Formulas 1 and 2 above, L1 and L2 are each independently oxygen, and
in Formula 3 above, M is tin.

10. The resist compound for photolithography of claim 1, wherein the organic molecule is represented by Formula 8: 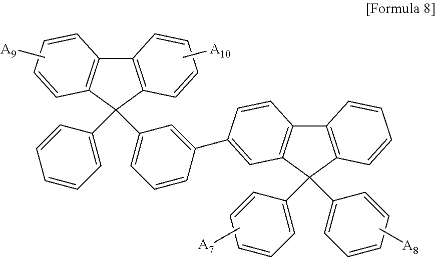
wherein in Formula 8 above, A7, A8, A9, and A10 are each independently the functional group represented by Formula 1 or Formula 2 above.

11. The resist compound for photolithography of claim 10, wherein in Formulas 1 and 2 above, L1 and L2 are each independently oxygen, and
in Formula 3 above, M is tin.

12. The resist compound for photolithography of claim 1, wherein the organic molecule is represented by Formula 9: 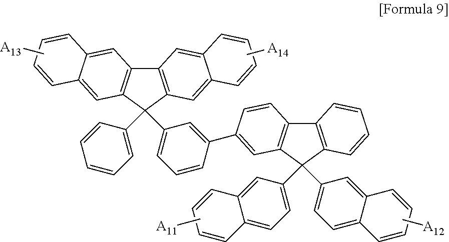
wherein in Formula 9 above, A11, A12, A13, and A14 are each independently the functional group represented by Formula 1 or Formula 2 above.

13. The resist compound for photolithography of claim 12, wherein in Formulas 1 and 2 above, L1 and L2 are each independently oxygen, and
in Formula 3 above, M is tin.

14. The resist compound for photolithography of claim 1, wherein the resist compound is a non-chemically amplified resist containing the organic molecule.

15. A method for manufacturing a semiconductor device, the method comprising forming a photoresist film on a substrate,
wherein the photoresist film includes a resist compound containing an organic molecule,
the organic molecule includes a core structure containing at least six benzene rings and a functional group bonded to at least one of the benzene rings, and
the functional group is represented by Formula 1 or Formula 2: 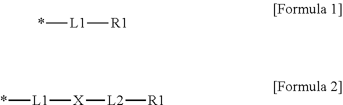
wherein in Formulas 1 and 2 above,
* is a portion bonded to a carbon of the at least one of the benzene rings,
L1 and L2 are each independently an alkylene group having 1 to 10 carbon atoms, or oxygen,
X is an alkylene group having 1 to 10 carbon atoms in which at least one hydrogen is substituted with a hydroxyl group (—OH),
R1 is an unsaturated hydrocarbon group having 2 to 10 carbon atoms with at least one C═C bond or at least one C≡C bond, or an unsaturated hydrocarbon group having 2 to 10 carbon atoms with at least one C═C bond and with at least one hydrogen substituted with a substituent represented by Formula 3: 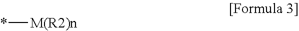
wherein in Formula 3 above, * is a portion bonded to a carbon of R1, M is a metal, R2 is an aryl group having 6 to 30 carbon atoms, and n is an integer of 1 to 3.

16. The method of claim 15, wherein in Formulas 1 and 2, R1 is represented by Formula 4-1, Formula 4-2, or Formula 4-3: 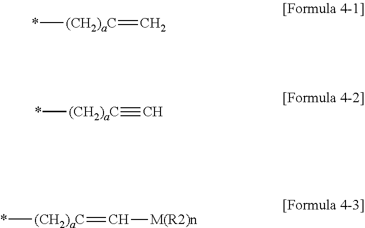
wherein in Formulas 4-1, 4-2, and Formula 4-3, * is a portion bonded to L1 of Formula 1 above or L2 of Formula 2 above, and a is an integer of 1 to 8, and
in Formula 4-3 above, M, R2, and n are as defined in Formula 3 above.

17. The method of claim 16, wherein in Formulas 1 and 2 above, L1 and L2 are each independently oxygen, and
in Formula 3 above, M is tin.

18. The method of claim 15, further comprising performing an exposure process on the photoresist film,
wherein the exposure process is performed using deep UV, extreme UV, or e-beams.

19. The method of claim 18, wherein the photoresist film comprises a first portion exposed by the exposure process and a second portion not exposed by the exposure process, and
in the first portion of the photoresist film, the resist compound comprises a structure in which the organic molecules are cross-linked.

20. The method of claim 15, further comprising forming an underlayer between the substrate and the photoresist film,
wherein the underlayer includes cross-linked poly(4-hydroxystyrene) (PHS).
